(19) United States
(12) Patent Application Publication
(10) Pub. No.: US 2013/0225830 A1
(43) Pub. Date: Aug. 29, 2013

(54) POLYMORPHS OF FEBUXOSTAT

(71) Applicants: Ehud Marom, Kfar Saba (IL); Shai Rubnov, Tel Aviv (IL)

(72) Inventors: Ehud Marom, Kfar Saba (IL); Shai Rubnov, Tel Aviv (IL)

(73) Assignee: MAPI PHARMA LIMITED, Ness Ziona (IL)

(21) Appl. No.: 13/881,311

(22) PCT Filed: Mar. 17, 2011
(86) PCT No.: PCT/IL11/00258

§ 371 Date: Apr. 24, 2013

Related U.S. Application Data
(60) Provisional application No. 61/407,465, filed on Oct. 28, 2010.

(51) Int. Cl. C07D 277/56

(52) U.S. Cl. CPC C07D 277/56
USPC 548/201

(57) ABSTRACT

The present invention provides new crystalline forms of febuxostat, pharmaceutical compositions comprising same, methods for their preparation and use thereof in treating hyperuricaemia.

### FIELD OF THE INVENTION

The present invention relates to new crystalline forms of febuxostat, pharmaceutical compositions comprising same, and use thereof in treating hyperuricaemia.

### BACKGROUND OF THE INVENTION

Febuxostat is a potent, selective, non-purine inhibitor of xanthine oxidase. Febuxostat has been approved for the treatment of chronic hyperuricaemia in conditions in which urate deposition has occurred, such as gouty arthritis.

Febuxostat is chemically named 2-(3-cyano-4-isobutoxyphenyl)-4-methyl-5-thiazolecarboxylic acid, and is represented by the following chemical structure:

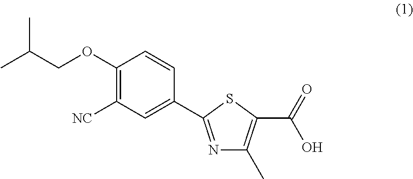 [SMILES: Cc1nc(-c2ccc(OCC(C)C)c(C#N)c2)sc1C(=O)O]

Febuxostat and processes for its preparation are disclosed in EP 0513379, JP 1993500083, U.S. Pat. No. 5,614,520 and WO 92/09279, JP 10-045733, JP 10-139770, JP 1994345724 (JP 6-345724), in publications in Heterocycles, 1998, 47: 857-864 and Org. Lett., 2009, 11(8): 1733-1736, and in PCT international patent application PCT/IL2010/000807 to some of the inventors of the present invention.

A new crystalline or amorphous form of a compound may possess physical properties that differ from, and are advantageous over, those of other crystalline or amorphous forms. These include, packing properties such as molar volume, density and hygroscopicity; thermodynamic properties such as melting temperature, vapor pressure and solubility; kinetic properties such as dissolution rate and stability under various storage conditions; surface properties such as surface area, wettability, interfacial tension and shape; mechanical properties such as hardness, tensile strength, compactibility, handling, flow and blend; and filtration properties. Variations in any one of these properties affect the chemical and pharmaceutical processing of a compound as well as its bioavailability and may render the new form advantageous for medical use.

EP 0513379 discloses a polymorph of febuxostat having a melting point of about 238-239° C. (decomposed).

U.S. Pat. No. 6,225,474 and U.S. Pat. No. 7,361,676 disclose six polymorphs of febuxostat, five crystalline polymorphs designated Forms A, B, C, D, and G and one amorphous form. Form A is characterized by the following X-ray diffraction peaks at about 6.62, 7.18, 12.80, 13.26, 16.48, 19.58, 21.92, 22.68, 25.84, 26.70, 29.16 and 36.70 2θ°; Another process for the preparation of Form A is disclosed in WO 2011/007895; Form B is characterized by the following X-ray diffraction peaks at about 6.76, 8.08, 9.74, 11.50, 12.22, 13.56, 15.76, 16.20, 17.32, 19.38, 21.14, 21.56, 23.16, 24.78, 25.14, 25.72, 26.12, 26.68, 27.68 and 29.36 2θ°; Form C is characterized by the following X-ray diffraction peaks at about 6.62, 10.82, 13.36, 15.52, 16.74, 17.40, 18.00, 18.70, 20.16, 20.62, 21.90, 23.50, 24.78, 25.18, 34.08, 36.72 and 38.04 2θ°; Form D (methanolate) is characterized by the following X-ray diffraction peaks at about 8.32, 9.68, 12.92, 16.06, 17.34, 19.38, 21.56, 24.06, 26.00, 30.06, 33.60 and 40.34 2θ°; and Form G (hydrate) is characterized by the following X-ray diffraction peaks at about 6.86, 8.36, 9.60, 11.76, 13.74, 14.60, 15.94, 16.74, 17.56, 20.00, 21.26, 23.72, 24.78, 25.14, 25.74, 26.06, 26.64, 27.92, 28.60, 29.66 and 29.98 2θ°.

CN 101386605 discloses a crystalline form of febuxostat designated as Form K, the form is characterized by the following X-ray diffraction peaks between 5.44 and 5.84, between 7.60 and 8.00, between 11.18 and 11.58, between 11.50 and 11.90, between 12.34 and 12.74, between 12.54 and 12.94, between 16.98 and 17.38, and between 25.92 and 26.32 2θ°.

CN 101412700 discloses a crystalline form of febuxostat which is characterized by the following X-ray diffraction peaks at 5.54±0.2, 5.66±0.2, 7.82±0.2, 11.48±0.2, 12.62±0.2, 16.74±0.2, 17.32±0.2, 18.04±0.2, 18.34±0.2, 20.40±0.2, 23.74±0.2, 25.76±0.2, and 26.04±0.2 2θ°.

WO 2008/067773 and CN 101474175 disclose three crystalline forms of febuxostat designated Forms H, I and J. Form H is characterized by the following X-ray diffraction peaks at about 6.71, 7.19, 10.03, 11.10, 12.96, 13.48, 15.78, 17.60 and 22.15 2θ°. Form I is characterized by the following X-ray diffraction peaks at about 3.28, 6.58, 12.70, 13.34, 19.97, 24.26, and 25.43 2θ°. Form J is characterized by the following X-ray diffraction peaks at about 3.07, 12.25, 13.16, 25.21, and 26.86 2θ°.

Other crystalline forms of febuxostat are described in CN 101928260, WO 2010/144685, CN 101891703, CN 101891702, CN 101759656, CN 101857578, CN 101824005, CN 101824007, CN 101824006, CN 101817801, CN 101805310, CN 101768136, CN 101768150, CN 101759656, CN 101684108, CN 101684107, CN 101671314, CN 101671315, CN 101648926, and CN 101139325.

There remains an unmet need for additional solid state forms of febuxostat having good physiochemical properties, desirable bioavailability, and advantageous pharmaceutical parameters.

### SUMMARY OF THE INVENTION

The present invention provides new crystalline forms of febuxostat, including anhydrous and solvated forms of febuxostat, pharmaceutical compositions comprising said forms, methods for their preparation and use thereof in treating hyperuricaemia.

The present invention is based in part on the unexpected finding that the new forms disclosed herein possess advantageous physicochemical properties which render their processing as medicaments beneficial. The forms of the present invention have good bioavailability as well as desirable hygroscopicity and stability characteristics enabling their incorporation into a variety of different formulations particularly suitable for pharmaceutical utility. Furthermore, anhydrous febuxostat (Form IX) of the present invention shows improved solubility at colon-simulated media (pHs=6.8-7.4) and intestinal fluids, thus indicating possible improved bioavailability.

According to one aspect, the present invention provides a crystalline form of febuxostat hydrate (Form II) having an X-ray powder diffraction pattern with diffraction peaks at 2-theta values at about 4.8±0.1, 6.9±0.1, 8.3±0.1, 9.6±0.1, 11.7±0.1, 13.7±0.1, 15.6±0.1, 16.7±0.1, 17.6±0.1, 19.9±0.1, 23.7±0.1, 25.2±0.1, 28.7±0.1, 30.0±0.1 and 34.3±0.1.

In one embodiment, the present invention provides a crystalline febuxostat hydrate (Form II) having an X-ray powder diffraction pattern substantially as shown in FIG. 1. In another embodiment, the crystalline febuxostat hydrate (Form II) is characterized by a DSC profile substantially as shown in FIG. 2. In another embodiment, the crystalline febuxostat hydrate (Form II) is characterized by a TGA profile substantially as shown in FIG. 3. In yet another embodiment, the crystalline febuxostat hydrate (Form II) is characterized by an IR spectrum substantially as shown in FIG. 4. In other embodiments, the crystalline febuxostat hydrate (Form II) has an IR spectrum with characteristic peaks at about 658±4, 725±4, 766±4, 824±4, 912±4, 956±4, 1010±4, 1042±4, 1114±4, 1164±4, 1216±4, 1286±4, 1323±4, 1369±4, 1393±4, 1425±4, 1467±4, 1508±4, 1601±4, 1679±4, 1698±4, 2222±4, 2872±4, and 2958±4 cm⁻¹. In certain embodiments, the crystalline febuxostat hydrate (Form II) is characterized by a FT-Raman spectrum substantially as shown in FIG. 5. In various embodiments, the FT-Raman spectrum of crystalline febuxostat hydrate (Form II) has characteristic peaks at about 1028±4, 1050±4, 1175±4, 1303±4, 1328±4, 1375±4, 1431±4, 1513±4, 1578±4, 1607±4, 2232±4, and 2930±4 cm⁻¹.

In certain embodiments, the present invention provides a process for preparing crystalline febuxostat hydrate (Form II), the process comprising the steps of:
(a) dissolving febuxostat in a solvent or a mixture of solvents selected from THF, THF:MeOH, THF:EtOH, THF:IPA, THF:1-Butanol, and THF:iPrOAc; and
(b) slowly evaporating the solvent or mixture of solvents so as to precipitate crystalline febuxostat hydrate (Form II).

In some embodiments, the solvents in the mixture of solvents are at a volume ratio of 1:1.

According to another aspect, the present invention provides a crystalline febuxostat N-methylpyrrolidone (i.e. NMP) solvate (Form IV) having an X-ray powder diffraction pattern with diffraction peaks at 2-theta values of about 4.0±0.1, 4.9±0.1, 6.4±0.1, 6.9±0.1, 7.5±0.1, 8.0±0.1, 8.3±0.1, 10.1±0.1, 10.7±0.1, 11.7±0.1, 12.3±0.1, 14.0±0.1, 16.0±0.1, 16.7±0.1, 17.2±0.1, 17.6±0.1, 18.8±0.1, 20.1±0.1, 20.9±0.1, 21.6±0.1, 23.2±0.1, 23.6±0.1, 25.2±0.1, and 26.2±0.1.

In some embodiments, the present invention provides a crystalline febuxostat NMP solvate (Form IV) having an X-ray powder diffraction pattern substantially as shown in FIG. 6. In another embodiment, the crystalline febuxostat NMP solvate (Form IV) is characterized by a DSC profile substantially as shown in FIG. 7. In another embodiment, the crystalline febuxostat NMP solvate (Form IV) is characterized by a TGA profile substantially as shown in FIG. 8. In yet another embodiment, the crystalline febuxostat NMP solvate (Form IV) is characterized by an IR spectrum substantially as shown in FIG. 9. In other embodiments, the crystalline febuxostat NMP solvate (Form IV) has an IR spectrum with characteristic peaks at about 658±4, 725±4, 762±4, 826±4, 907±4, 952±4, 1010±4, 1037±4, 1129±4, 1164±4, 1217±4, 1283±4, 1319±4, 1370±4, 1397±4, 1426±4, 1467±4, 1509±4, 1604±4, 1682±4, 2227±4, 2872±4, and 2962±4 cm⁻¹. In certain embodiments, the crystalline febuxostat NMP solvate (Form IV) is characterized by a FT-Raman spectrum substantially as shown in FIG. 10. In various embodiments, the FT-Raman spectrum of crystalline febuxostat NMP solvate (Form IV) has characteristic peaks at about 155±4, 197±4, 326±4, 409±4, 467±4, 531±4, 836±4, 913±4, 1028±4, 1110±4, 1175±4, 1286±4, 1332±4, 1374±4, 1431±4, 1512±4, 1606±4, 1842±4, 1898±4, 2070±4, 2116±4, and 2232±4 cm⁻¹.

In certain embodiments, the present invention provides a process for preparing crystalline febuxostat NMP solvate (Form IV), the process comprising the steps of:
(a) dissolving febuxostat in a solvent or a mixture of solvents selected from NMP, 2-MeTHF:NMP, DMF:NMP, and NMP:THF, optionally under heat; and
(b) cooling the solution obtained in step (a) so as to precipitate crystalline febuxostat NMP solvate (Form IV).

In some embodiments, the solvents in the mixture of solvents are at a volume ratio of 1:1.

According to another aspect, the present invention provides a crystalline febuxostat NMP solvate (Form VI) having an X-ray powder diffraction pattern with diffraction peaks at 2-theta values of about 4.1±0.1, 7.0±0.1, 7.6±0.1, 8.3±0.1, 10.0±0.1, 11.4±0.1, 12.5±0.1, 13.7±0.1, 14.1±0.1, 15.4±0.1, 17.1±0.1, 17.6±0.1, 19.6±0.1, 21.5±0.1, 23.0±0.1, 24.9±0.1, 25.3±0.1, 25.6±0.1, 26.2±0.1, 27.1±0.1, and 29.9±0.1.

In some embodiments, the present invention provides a crystalline febuxostat NMP solvate (Form VI) having an X-ray powder diffraction pattern substantially as shown in FIG. 11. In another embodiment, the crystalline febuxostat NMP solvate (Form VI) is characterized by a DSC profile substantially as shown in FIG. 12. In another embodiment, the crystalline febuxostat NMP solvate (Form VI) is characterized by a TGA profile substantially as shown in FIG. 13. In yet another embodiment, the crystalline febuxostat NMP solvate (Form VI) is characterized by an IR spectrum substantially as shown in FIG. 14. In other embodiments, the crystalline febuxostat NMP solvate (Form VI) has an IR spectrum with characteristic peaks at about 657±4, 716±4, 745±4, 764±4, 824±4, 903±4, 948±4, 1007±4, 1042±4, 1091±4, 1128±4, 1170±4, 1223±4, 1262±4, 1295±4, 1372±4, 1393±4, 1428±4, 1471±4, 1508±4, 1604±4, 1682±4, 1699±4, 1728±4, 2222±4, 2868±4, and 2962±4 cm⁻¹. In certain embodiments, the crystalline febuxostat NMP solvate (Form VI) is characterized by a FT-Raman spectrum substantially as shown in FIG. 15. In particular embodiments, the FT-Raman spectrum of crystalline febuxostat NMP solvate (Form VI) has characteristic peaks at about 1028±4, 1317±4, 1374±4, 1434±4, 1512±4, 1606±4, and 2229±4 cm⁻¹.

In further embodiments, the present invention provides a process for preparing crystalline febuxostat NMP solvate (Form VI), the process comprising the steps of:
(a) dissolving febuxostat in NMP, optionally under heat; and
(b) adding an anti-solvent selected from water and ACN so as to precipitate crystalline febuxostat NMP solvate (Form VI).

According to yet another aspect, the present invention provides a crystalline febuxostat DMSO solvate (Form V) having an X-ray powder diffraction pattern with diffraction peaks at 2-theta values of about 7.1±0.1, 10.6±0.1, 11.7±0.1, 13.8±0.1, 14.3±0.1, 15.2±0.1, 16.2±0.1, 16.9±0.1, 17.2±0.1, 19.4±0.1, 21.0±0.1, 21.6±0.1, 21.8±0.1, 22.1±0.1, 22.5±0.1, 22.7±0.1, 23.5±0.1, 24.8±0.1, 26.4±0.1, and 28.7±0.1.

In some embodiments, the present invention provides a crystalline febuxostat DMSO solvate (Form V) having an X-ray powder diffraction pattern substantially as shown in any of FIG. 16 or 39. In another embodiment, the crystalline febuxostat DMSO solvate (Form V) is characterized by a DSC profile substantially as shown in any of FIG. 17 or 40. In another embodiment, the crystalline febuxostat DMSO solvate (Form V) is characterized by a TGA profile substantially as shown in any of FIG. 18 or 41. In yet another embodiment, the crystalline febuxostat DMSO solvate (Form V) is characterized by an IR spectrum substantially as shown in FIG. 19. In other embodiments, the crystalline febuxostat DMSO solvate (Form V) has an IR spectrum with characteristic peaks at about 653±4, 706±4, 743±4, 766±4, 827±4, 881±4, 907±4, 951±4, 1005±4, 1106±4, 1164±4, 1274±4, 1315±4, 1368±4, 1389±4, 1426±4, 1450±4, 1509±4, 1573±4, 1604±4, 1679±4, 2227±4, 2868±4, and 2966±4 cm⁻¹. In certain embodiments, the crystalline febuxostat DMSO solvate (Form V) is characterized by a FT-Raman spectrum substantially as shown in FIG. 20. In particular embodiments, the FT-Raman spectrum of crystalline febuxostat DMSO solvate (Form V) has characteristic peaks at about 288±4, 337±4, 395±4, 433±4, 531±4, 578±4, 672±4, 708±4, 1041±4, 1323±4, 1371±4, 1452±4, 1512±4, 1574±4, 1609±4, and 1690±4 cm⁻¹.

In further embodiments, the present invention provides a process for preparing crystalline febuxostat DMSO solvate (Form V), the process comprising the steps of:
(a) dissolving febuxostat in a solvent or a mixture of solvents selected from DMSO, 2-MeTHF:DMSO, DMF:DMSO, and NMP:DMSO, optionally under heat; and
(b) cooling the solution obtained in step (a) so as to precipitate crystalline febuxostat DMSO solvate (Form V).

In some embodiments, the solvents in the mixture of solvents are at a volume ratio of 1:1. In other embodiments, the process for preparing crystalline febuxostat DMSO solvate (Form V) further comprises the steps of:
(c) separating the precipitate by vacuum filtration;
(d) washing the precipitate with acetonitrile (ACN); and
(e) drying the precipitate under vacuum to obtain febuxostat DMSO solvate (Form V).

According to another aspect, the present invention provides a crystalline febuxostat DMSO solvate (Form VII) having an X-ray powder diffraction pattern with diffraction peaks at 2-theta values of about 4.0±0.1, 7.2±0.1, 8.0±0.1, 11.4±0.1, 13.6±0.1, 13.9±0.1, 14.7±0.1, 17.1±0.1, 17.8±0.1, 20.5±0.1, 21.5±0.1, 22.7±0.1, 23.0±0.1, 25.2±0.1, 26.3±0.1, and 27.8±0.1.

In some embodiments, the present invention provides a crystalline febuxostat DMSO solvate (Form VII) having an X-ray powder diffraction pattern substantially as shown in FIG. 21. In another embodiment, the crystalline febuxostat DMSO solvate (Form VII) is characterized by a DSC profile substantially as shown in FIG. 22. In another embodiment, the crystalline febuxostat DMSO solvate (Form VII) is characterized by a TGA profile substantially as shown in FIG. 23. In yet another embodiment, the crystalline febuxostat DMSO solvate (Form VII) is characterized by an IR spectrum substantially as shown in FIG. 24. In other embodiments, the crystalline febuxostat DMSO solvate (Form VII) has an IR spectrum with characteristic peaks at about 653±4, 702±4, 743±4, 765±4, 827±4, 878±4, 951±4, 1009±4, 1106±4, 1160±4, 1274±4, 1315±4, 1368±4, 1389±4, 1422±4, 1450±4, 1509±4, 1605±4, 1680±4, 2222±4, 2872±4, and 2962±4 cm⁻¹. In certain embodiments, the crystalline febuxostat DMSO solvate (Form VII) is characterized by a FT-Raman spectrum substantially as shown in FIG. 25. In particular embodiments, the FT-Raman spectrum of crystalline febuxostat DMSO solvate (Form VII) has characteristic peaks at about 357±4, 467±4, 531±4, 578±4, 675±4, 839±4, 1028±4, 1110±4, 1175±4, 1286±4, 1323±4, 1371±4, 1449±4, 1512±4, 1571±4, 1609±4, 1693±4, 1842±4, 2081±4, 2116±4, 2227±4, 2923±4, and 3502±4 cm⁻¹.

In further embodiments, the present invention provides a process for preparing crystalline febuxostat DMSO solvate (Form VII), the process comprising the steps of:
(a) dissolving febuxostat in DMSO, optionally under heat; and
(b) adding an anti-solvent, wherein the anti-solvent is ACN so as to precipitate crystalline febuxostat DMSO solvate (Form VII).

According to another aspect, the present invention provides a crystalline febuxostat anhydrous (Form VIII) having an X-ray powder diffraction pattern with diffraction peaks at 2-theta values of about 3.6±0.1, 7.1±0.1, 12.4±0.1, 13.3±0.1, 17.6±0.1, 23.1±0.1, 25.2±0.1, 27.0±0.1, and 27.6±0.1.

In some embodiments, the present invention provides a crystalline anhydrous febuxostat (Form VIII) having an X-ray powder diffraction pattern substantially as shown in FIG. 26. In another embodiment, the crystalline anhydrous febuxostat (Form VIII) is characterized by a DSC profile substantially as shown in FIG. 27. In another embodiment, the crystalline anhydrous febuxostat (Form VIII) is characterized by a TGA profile substantially as shown in FIG. 28. In yet another embodiment, the crystalline anhydrous febuxostat (Form VIII) is characterized by an IR spectrum substantially as shown in FIG. 29. In other embodiments, the crystalline anhydrous febuxostat (Form VIII) has an IR spectrum with characteristic peaks at about 660±4, 725±4, 764±4, 824±4, 878±4, 910±4, 930±4, 1012±4, 1037±4, 1116±4, 1172±4, 1283±4, 1328±4, 1371±4, 1385±4, 1425±4, 1467±4, 1510±4, 1604±4, 1653±4, 1683±4, 2231±4, 2868±4, and 2958±4 cm⁻¹. In certain embodiments, the crystalline anhydrous febuxostat (Form VIII) is characterized by a FT-Raman spectrum substantially as shown in FIG. 30. In particular embodiments, the FT-Raman spectrum of crystalline anhydrous febuxostat (Form VIII) has characteristic peaks at about 155±4, 239±4, 288±4, 347±4, 402±4, 467±4, 538±4, 605±4, 672±4, 748±4, 839±4, 913±4, 1009±4, 1100±4, 1175±4, 1286±4, 1326±4, 1374±4, 1434±4, 1512±4, 1609±4, 1664±4, 1768±4, 1864±4, 1898±4, 1973±4, 2070±4, 2235±4, 2272±4, and 2390±4 cm⁻¹.

In further embodiments, the present invention provides a process for preparing crystalline anhydrous febuxostat (Form VIII), the process comprising the steps of:
(a) heating febuxostat to melt under vacuum; and
(b) cooling the melted febuxostat obtained in step (a), so as to provide crystalline anhydrous febuxostat (Form VIII).

In some embodiments, the cooling in step (b) is selected from fast cooling and slow cooling. Each possibility represents a separate embodiment of the invention.

According to another aspect, the present invention provides a crystalline anhydrous febuxostat (Form IX) having an X-ray powder diffraction pattern with diffraction peaks at 2-theta values of about 4.6±0.1, 6.1±0.1, 7.3±0.1, 9.2±0.1, 11.6±0.1, 13.3±0.1, 16.3±0.1, 17.3±0.1, 18.5±0.1, 23.0±0.1, 25.7±0.1, 26.5±0.1 and 28.3±0.1.

In some embodiments, the present invention provides a crystalline anhydrous febuxostat (Form IX) having an X-ray powder diffraction pattern substantially as shown in any of FIG. 31 or 42. In another embodiment, the crystalline anhydrous febuxostat (Form IX) is characterized by a DSC profile substantially as shown in any of FIG. 32 or 43. In another embodiment, the crystalline anhydrous febuxostat (Form IX) is characterized by a TGA profile substantially as shown in any of FIG. 33 or 44. In yet another embodiment, the crystalline anhydrous febuxostat (Form IX) is characterized by an IR spectrum substantially as shown in FIG. 34. In other embodiments, the crystalline anhydrous febuxostat (Form IX) has an IR spectrum with characteristic peaks at about 657±4, 715±4, 764±4, 825±4, 874±4, 911±4, 952±4, 1010±4, 1037±4, 1114±4, 1168±4, 1281±4, 1328±4, 1370±4, 1389±4, 1427±4, 1450±4, 1511±4, 1606±4, 1687±4, 2235±4, 2868±4 and 2962±4 cm⁻¹. In certain embodiments, the crystalline anhydrous febuxostat (Form IX) is characterized by a FT-Raman spectrum substantially as shown in FIG. 35. In particular embodiments, the FT-Raman spectrum of crystalline anhydrous febuxostat (Form IX) has characteristic peaks at about 392±4, 467±4, 585±4, 748±4, 1047±4, 1175±4, 1332±4, 1374±4, 1431±4, 1512±4, 1609±4, 1842±4, 1892±4, 1973±4, 2081±4, and 2235±4 cm⁻¹.

In further embodiments, the present invention provides a process for preparing crystalline anhydrous febuxostat (Form IX), the process comprising the steps of:
(a) dissolving febuxostat in a solvent selected from MeOH, MEK, acetone, and EtOAc; and
(b) rapidly evaporating the solvent so as to precipitate crystalline anhydrous febuxostat (Form IX).

In one embodiment, the solvent in step (a) is EtOAc. In some embodiments, the evaporation in step (b) is performed using rotary evaporator, preferably at a temperature of 50° C. or below. In other embodiments, the process for preparing crystalline anhydrous febuxostat (Form IX) further comprises the step of drying the febuxostat (Form IX) obtained in step (b) under vacuum.

According to another aspect, the present invention provides a crystalline form of febuxostat hydrate (Form XI) having an X-ray powder diffraction pattern with diffraction peaks at 2-theta values at about 4.9±0.1, 6.2±0.1, 6.8±0.1, 8.2±0.1, 9.7±0.1, 11.6±0.1, 12.2±0.1, 13.6±0.1, 15.8±0.1, 16.3±0.1, 17.5±0.1, 19.4±0.1, 20.5±0.1, 21.3±0.1, 21.5±0.1, 23.2±0.1, 24.8±0.1, 25.2±0.1, 25.8±0.1, 26.2±0.1, 26.8±0.1, 27.8±0.1, 29.2±0.1 and 29.8±0.1.

In one embodiment, the present invention provides a crystalline febuxostat hydrate (Form XI) having an X-ray powder diffraction pattern substantially as shown in FIG. 36. In another embodiment, the crystalline febuxostat hydrate (Form XI) is characterized by a DSC profile substantially as shown in FIG. 37. In another embodiment, the crystalline febuxostat hydrate (Form XI) is characterized by a TGA profile substantially as shown in FIG. 38.

In certain embodiments, the present invention provides a process for preparing crystalline febuxostat hydrate (Form XI), the process comprising the steps of:
(a) dissolving febuxostat in THF to obtain a clear solution;
(b) slowly evaporating the THF to obtain a precipitate; and
(c) drying the precipitate in vacuum so as to provide crystalline febuxostat hydrate (Form XI).

In some embodiments, the step of drying the precipitate is conducted at about 40° C.

It has unexpectedly been found that crystalline anhydrous febuxostat (Form IX) shows improved solubility at colon-simulated media (pHs=6.8-7.4) and intestinal fluids. As the major site of absorption for febuxostat is the colon, this suggests a possible improved bioavailability.

In certain embodiments, the present invention provides a pharmaceutical composition comprising as an active ingredient any one of the febuxostat forms of the present invention, and a pharmaceutically acceptable carrier. In one embodiment, the present invention provides a pharmaceutical composition comprising as an active ingredient the crystalline anhydrous febuxostat (Form IX) of the present invention, and a pharmaceutically acceptable carrier. In another embodiment, the pharmaceutical composition comprises as an active ingredient crystalline febuxostat NMP solvate (Form IV). In yet another embodiment, the pharmaceutical composition comprises as an active ingredient crystalline febuxostat NMP solvate (Form VI). In additional embodiments, the pharmaceutical composition comprises as an active ingredient crystalline febuxostat DMSO solvate (Form VII). In further embodiments, the pharmaceutical composition comprises as an active ingredient crystalline anhydrous febuxostat (Form VIII).

In further embodiments, the present invention provides a pharmaceutical composition comprising as an active ingredient a single crystalline febuxostat form of the present invention, and a pharmaceutically acceptable carrier. In one embodiment, the single crystalline febuxostat form is anhydrous febuxostat (Form IX). In other embodiments, the single crystalline febuxostat form is any one of forms IV, VI, VII or VIII. Each possibility represents a separate embodiment of the present invention.

In a particular embodiment, the pharmaceutical composition is in the form of a tablet.

In various embodiments, the present invention provides the pharmaceutical composition as disclosed herein for use in treating hyperuricaemia.

In some embodiments, the present invention provides a method of treating hyperuricaemia comprising administering to a subject in need thereof an effective amount of a pharmaceutical composition comprising any one of the febuxostat forms of the present invention. In particular embodiments, the present invention provides a method of treating hyperuricaemia comprising administering to a subject in need thereof an effective amount of a pharmaceutical composition comprising any one of febuxostat forms IX, IV, VI, VII or VIII of the present invention. Each possibility represents a separate embodiment of the present invention.

In certain embodiments, the subject is a mammal, for example a human.

In additional embodiments, the present invention provides the use of any one of the febuxostat forms of the present invention for treating hyperuricaemia. In further embodiments, the present invention provides the use of any one of febuxostat forms IX, IV, VI, VII or VIII of the present invention for treating hyperuricaemia. Each possibility represents a separate embodiment of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 illustrates a characteristic X-ray diffraction pattern of febuxostat hydrate (Form II).

FIG. 2 illustrates a characteristic Differential Scanning calorimetry (DSC) profile of febuxostat hydrate (Form II).

FIG. 3 illustrates a characteristic Thermogravimetric analysis (TGA) profile of febuxostat hydrate (Form II).

FIG. 4 illustrates a characteristic Infrared (IR) spectrum of febuxostat hydrate (Form II).

FIG. 5 illustrates a characteristic Fourier Transform—Raman (FT-Raman) spectrum of febuxostat hydrate (Form II).

FIG. 6 illustrates a characteristic X-ray diffraction pattern of febuxostat NMP solvate (Form IV).

FIG. 7 illustrates a characteristic Differential Scanning calorimetry (DSC) profile of febuxostat NMP solvate (Form IV).

FIG. 8 illustrates a characteristic Thermogravimetric analysis (TGA) profile of febuxostat NMP solvate (Form IV).

FIG. 9 illustrates a characteristic Infrared (IR) spectrum of febuxostat NMP solvate (Form IV).

FIG. 10 illustrates a characteristic Fourier Transform—Raman (FT-Raman) spectrum of febuxostat NMP solvate (Form IV).

FIG. 11 illustrates a characteristic X-ray diffraction pattern of febuxostat NMP solvate (Form VI).

FIG. 12 illustrates a characteristic Differential Scanning calorimetry (DSC) profile of febuxostat NMP solvate (Form VI).

FIG. 13 illustrates a characteristic Thermogravimetric analysis (TGA) profile of febuxostat NMP solvate (Form VI).

FIG. 14 illustrates a characteristic Infrared (IR) spectrum of febuxostat NMP solvate (Form VI).

FIG. 15 illustrates a characteristic Fourier Transform—Raman (FT-Raman) spectrum of febuxostat NMP solvate (Form VI).

FIG. 16 illustrates a characteristic X-ray diffraction pattern of febuxostat DMSO solvate (Form V).

FIG. 17 illustrates a characteristic Differential Scanning calorimetry (DSC) profile of febuxostat DMSO solvate (Form V).

FIG. 18 illustrates a characteristic Thermogravimetric analysis (TGA) profile of febuxostat DMSO solvate (Form V).

FIG. 19 illustrates a characteristic Infrared (IR) spectrum of febuxostat DMSO solvate (Form V).

FIG. 20 illustrates a characteristic Fourier Transform—Raman (FT-Raman) spectrum of febuxostat DMSO solvate (Form V).

FIG. 21 illustrates a characteristic X-ray diffraction pattern of febuxostat DMSO solvate (Form VII).

FIG. 22 illustrates a characteristic Differential Scanning calorimetry (DSC) profile of febuxostat DMSO solvate (Form VII).

FIG. 23 illustrates a characteristic Thermogravimetric analysis (TGA) profile of febuxostat DMSO solvate (Form VII).

FIG. 24 illustrates a characteristic Infrared (IR) spectrum of febuxostat DMSO solvate (Form VII).

FIG. 25 illustrates a characteristic Fourier Transform—Raman (FT-Raman) spectrum of febuxostat DMSO solvate (Form VII).

FIG. 26 illustrates a characteristic X-ray diffraction pattern of anhydrous febuxostat (Form VIII).

FIG. 27 illustrates a characteristic Differential Scanning calorimetry (DSC) profile of anhydrous febuxostat (Form VIII).

FIG. 28 illustrates a characteristic Thermogravimetric analysis (TGA) profile of anhydrous febuxostat (Form VIII).

FIG. 29 illustrates a characteristic Infrared (IR) spectrum of anhydrous febuxostat (Form VIII).

FIG. 30 illustrates a characteristic Fourier Transform—Raman (FT-Raman) spectrum of anhydrous febuxostat (Form VIII).

FIG. 31 illustrates a characteristic X-ray diffraction pattern of anhydrous febuxostat (Form IX).

FIG. 32 illustrates a characteristic Differential Scanning calorimetry (DSC) profile of anhydrous febuxostat (Form IX).

FIG. 33 illustrates a characteristic Thermogravimetric analysis (TGA) profile of anhydrous febuxostat (Form IX).

FIG. 34 illustrates a characteristic Infrared (IR) spectrum of anhydrous febuxostat (Form IX).

FIG. 35 illustrates a characteristic Fourier Transform—Raman (FT-Raman) spectrum of anhydrous febuxostat (Form IX).

FIG. 36 illustrates a characteristic X-ray diffraction pattern of febuxostat hydrate (Form XI).

FIG. 37 illustrates a characteristic Differential Scanning calorimetry (DSC) profile of febuxostat hydrate (Form XI).

FIG. 38 illustrates a characteristic Thermogravimetric analysis (TGA) profile of febuxostat hydrate (Form XI).

FIG. 39 illustrates a characteristic X-ray diffraction pattern of febuxostat DMSO solvate (Form V) scale-up.

FIG. 40 illustrates a characteristic Differential Scanning calorimetry (DSC) profile of febuxostat DMSO solvate (Form V) scale-up.

FIG. 41 illustrates a characteristic Thermogravimetric analysis (TGA) profile of febuxostat DMSO solvate (Form V) scale-up.

FIG. 42 illustrates a characteristic X-ray diffraction pattern of anhydrous febuxostat (Form IX) scale-up.

FIG. 43 illustrates a characteristic Differential Scanning calorimetry (DSC) profile of anhydrous febuxostat (Form IX) scale-up.

FIG. 44 illustrates a characteristic Thermogravimetric analysis (TGA) profile of anhydrous febuxostat (Form IX) scale-up.

FIG. 45 illustrates a characteristic dynamic vapor sorption (DVS) isotherm plot of anhydrous febuxostat (Form IX). Sorption (♦); Desorption (▪).

FIG. 46 illustrates a characteristic dynamic vapor sorption (DVS) isotherm plot of febuxostat hydrate (Form XI). Sorption (♦); Desorption (▪).

FIG. 47 illustrates a characteristic dynamic vapor sorption (DVS) isotherm plot of febuxostat DMSO solvate (Form V). Sorption (♦); Desorption (▪).

FIG. 48 illustrates a characteristic dynamic vapor sorption (DVS) isotherm plot of febuxostat Form G of U.S. Pat. No. 6,225,474. Sorption (♦); Desorption (▪).

FIG. 49 illustrates a characteristic X-ray diffraction pattern of febuxostat DMSO solvate (Form V) single crystal. Also shown for comparison is the X-ray diffraction pattern of febuxostat DMSO solvate (Form V) powder.

FIG. 50 illustrates the structure of a single crystal of febuxostat DMSO solvate (Form V).

### DETAILED DESCRIPTION OF THE INVENTION

The present invention is directed to novel crystalline forms of 2-(3-cyano-4-isobutoxyphenyl)-4-methyl-5-thiazolecarboxylic acid having structural formula (1).

The present invention is further directed to pharmaceutical compositions comprising the crystalline forms and a pharmaceutically acceptable carrier and their use in treating hyperuricaemia.

The present invention is further directed to methods of preparing the novel forms of febuxostat of the present invention.

Polymorphs are two or more solid state phases of the same chemical compound that possess different arrangement and/or conformation of the molecules. Different polymorphs of an active pharmaceutical compound can exhibit different physical and chemical properties such as color, stability, processability, dissolution and even bioavailability.

The identification and characterization of various polymorphs of a pharmaceutically active compound is therefore of great significance in obtaining medicaments with desired properties including a specific dissolution rate, milling properties, bulk density, thermal stability or shelf-life. The febuxostat forms of the present invention possess improved physicochemical characteristics including improved solubility at colon-simulated media and intestinal fluids (pH of 6.8-7.4). Furthermore, the febuxostat forms of the present invention are significantly less hygroscopic at the ICH recommended storage conditions and remain stable when stored over prolonged periods of time.

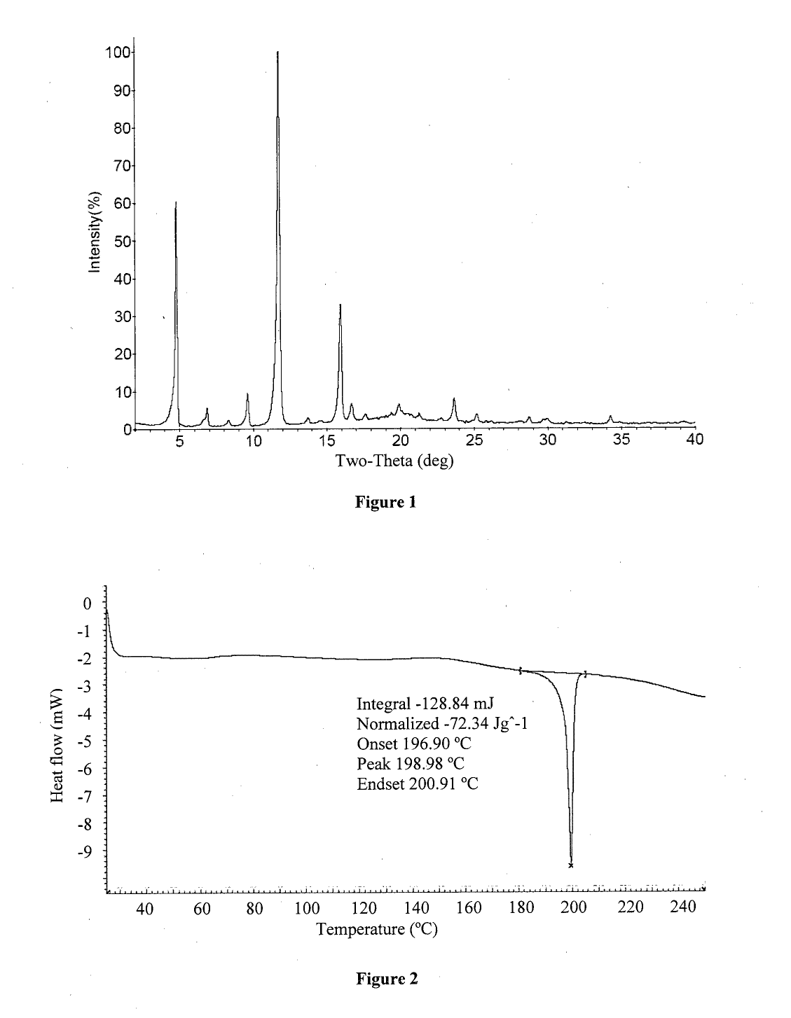

Provided herein is crystalline form of febuxostat hydrate (Form II) which is characterized by an X-ray diffraction pattern substantially as shown in FIG. 1 with peaks at 2-theta values of about 4.8±0.1, 6.9±0.1, 8.3±0.1, 9.6±0.1, 11.7±0.1, 13.7±0.1, 15.6±0.1, 16.7±0.1, 17.6±0.1, 19.9±0.1, 23.7±0.1, 25.2±0.1, 28.7±0.1, 30.0±0.1 and 34.3±0.1. The febuxostat hydrate (From II) is further characterized using various techniques including infrared absorption, Raman spectrometry, and thermal analysis (e.g. thermogravimetric analysis (TGA) and differential scanning calorimetry (DSC)).

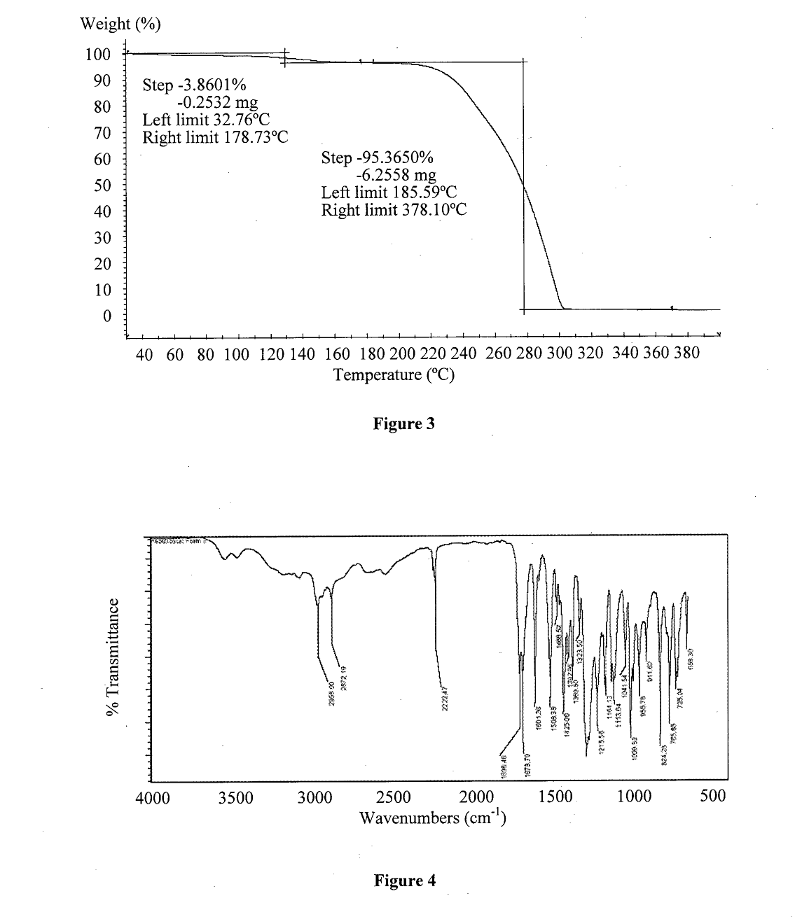

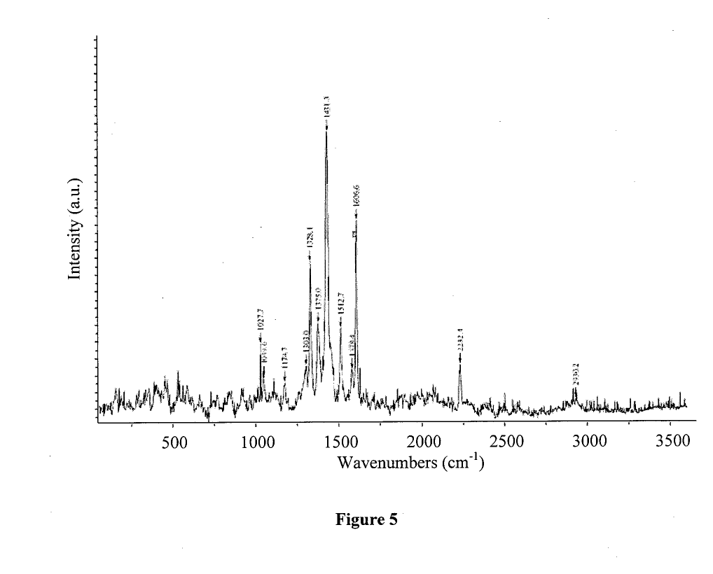

In some embodiments, the febuxostat (Form II) of the present invention is characterized by DSC and TGA profiles substantially as shown in FIGS. 2 and 3, respectively. The febuxostat (Form II) is further characterized by infrared spectrum substantially as shown in FIG. 4 with characteristic peaks at the following wavenumbers: about 658, about 725, about 766, about 824, about 912, about 956, about 1010, about 1042, about 1114, about 1164, about 1216, 1286, about 1323, about 1369, about 1393, about 1425, about 1467, about 1508, about 1601, about 1679, about 1698, about 2222, about 2872, and about 2958 cm⁻¹. The febuxostat (Form II) is characterized by FT-Raman substantially as shown in FIG. 5 with characteristic peaks at the following wavenumbers: about 1028, about 1050, about 1175, about 1303, about 1328, about 1375, about 1431, about 1513, about 1578, about 1607, about 2232, and about 2930 cm⁻¹.

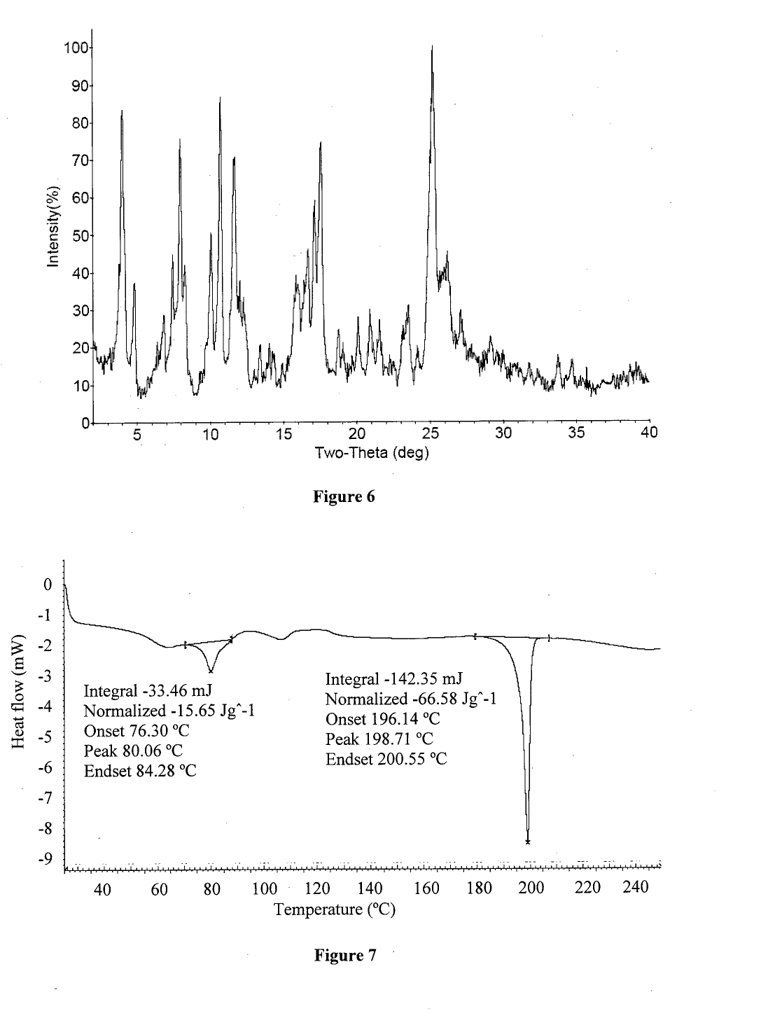

The present invention further provides a crystalline febuxostat NMP solvate (Form IV) which is characterized by an X-ray diffraction pattern substantially as shown in FIG. 6 with peaks at 2 theta values of about 4.0±0.1, 4.9±0.1, 6.4±0.1, 6.9±0.1, 7.5±0.1, 8.0±0.1, 8.3±0.1, 10.1±0.1, 10.7±0.1, 11.7±0.1, 12.3±0.1, 14.0±0.1, 16.0±0.1, 16.7±0.1, 17.2±0.1, 17.6±0.1, 18.8±0.1, 20.1±0.1, 20.9±0.1, 21.6±0.1, 23.2±0.1, 23.6±0.1, 25.2±0.1, and 26.2±0.1. The febuxostat NMP solvate (Form IV) is further characterized using various techniques including infrared absorption, Raman spectrometry, and thermal analysis (e.g. thermogravimetric analysis (TGA) and differential scanning calorimetry (DSC)).

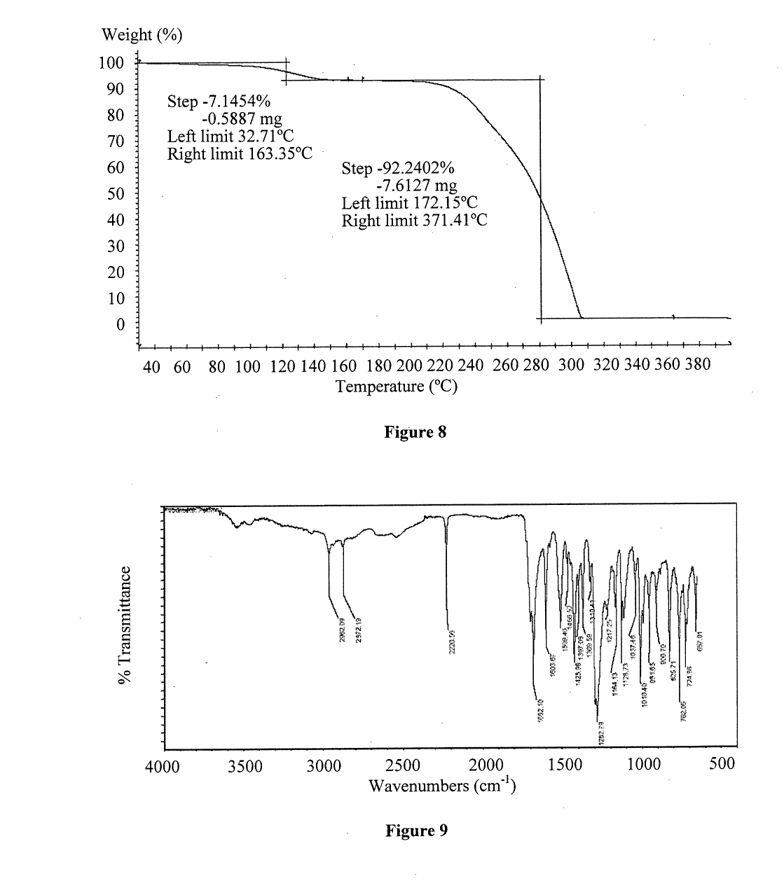

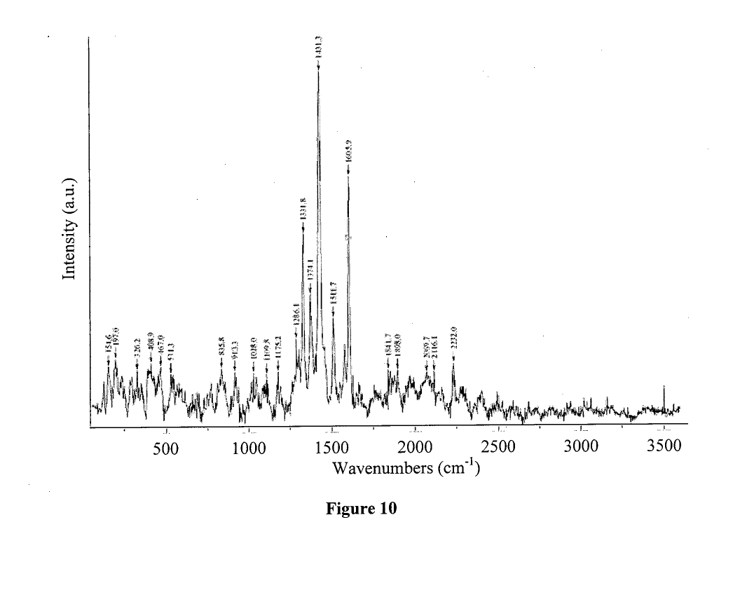

In certain embodiments, the febuxostat NMP solvate (Form IV) of the present invention is characterized by DSC and TGA profiles substantially as shown in FIGS. 7 and 8, respectively. The febuxostat (Form IV) is further characterized by Infrared spectrum substantially as shown in FIG. 9 with characteristic peaks at the following wavenumbers: about 658, about 725, about 762, about 826, about 907, about 952, about 1010, about 1037, about 1129, about 1164, about 1217, about 1283, about 1319, about 1370, about 1397, about 1426, about 1467, about 1509, about 1604, about 1682, about 2227, about 2872, and about 2962 cm⁻¹. The febuxostat (Form IV) is characterized by FT-Raman substantially as shown in FIG. 10 with characteristic peaks at the following wavenumbers: about 155, about 197, about 326, about 409, about 467, about 531, about 836, about 913, about 1028, about 1110, about 1175, about 1286, about 1332, about 1374, about 1431, about 1512, about 1606, about 1842, about 1898, about 2070, about 2116, and about 2232 cm⁻¹.

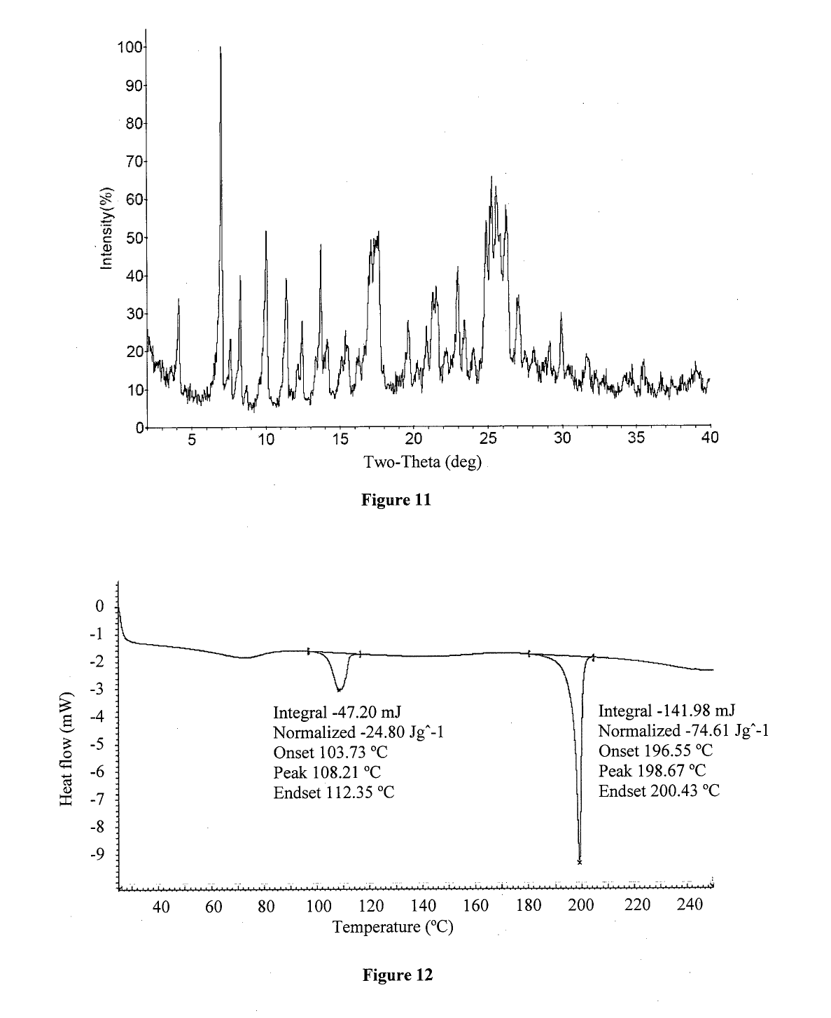

Further provided herein is a crystalline febuxostat NMP solvate (Form VI) which is characterized by an X-ray diffraction pattern substantially as shown in FIG. 11 with peaks at 2 theta values of about 4.1±0.1, 7.0±0.1, 7.6±0.1, 8.3±0.1, 10.0±0.1, 11.4±0.1, 12.5±0.1, 13.7±0.1, 14.1±0.1, 15.4±0.1, 17.1±0.1, 17.6±0.1, 19.6±0.1, 21.5±0.1, 23.0±0.1, 24.9±0.1, 25.3±0.1, 25.6±0.1, 26.2±0.1, 27.1±0.1, and 29.9±0.1. The febuxostat NMP solvate (Form VI) is further characterized using various techniques including infrared absorption, Raman spectrometry, and thermal analysis (e.g. thermogravimetric analysis (TGA) and differential scanning calorimetry (DSC)).

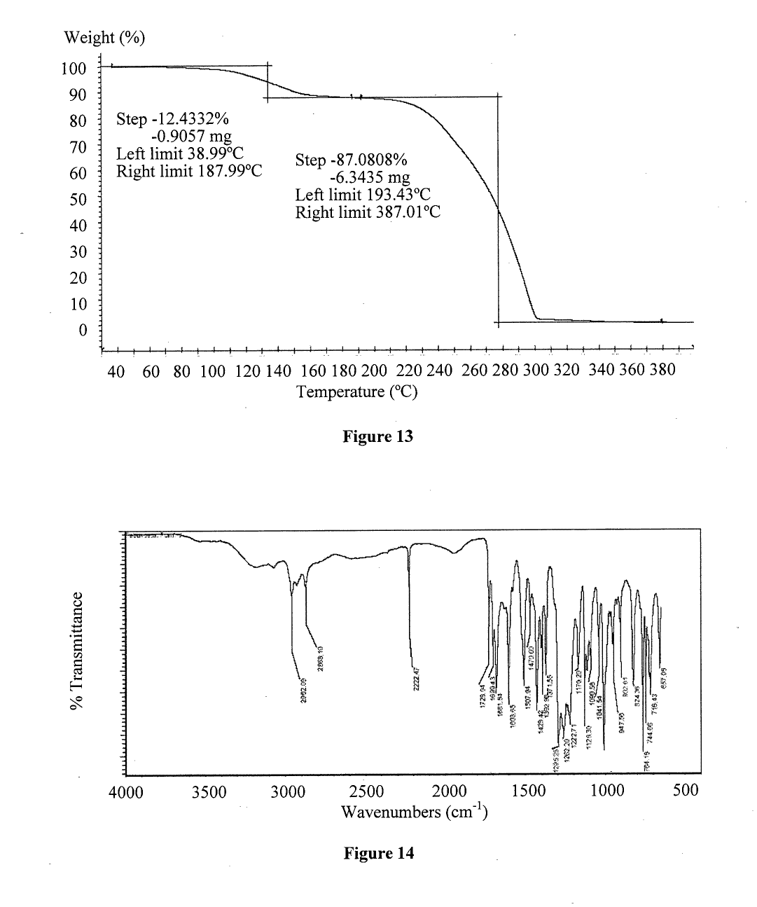

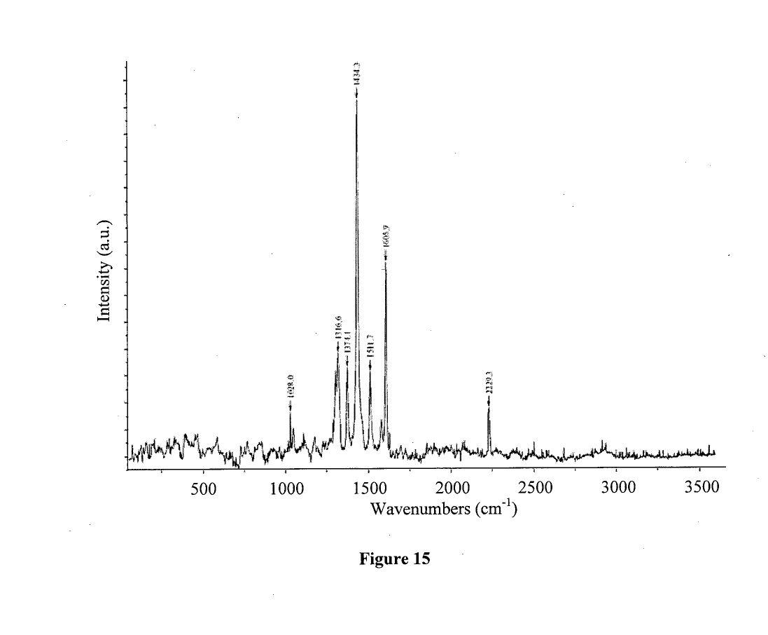

In various embodiments, the febuxostat NMP solvate (Form VI) of the present invention is characterized by DSC and TGA profiles substantially as shown in FIGS. 12 and 13, respectively. The febuxostat (Form VI) is further characterized by Infrared spectrum substantially as shown in FIG. 14 with characteristic peaks at the following wavenumbers: about 657, about 716, about 745, about 764, about 824, about 903, about 948, 1007, about 1042, about 1091, about 1128, about 1170, about 1223, about 1262, about 1295, about 1372, about 1393, about 1428, about 1471, about 1508, about 1604, about 1682, about 1699, about 1728, about 2222, about 2868, and about 2962 cm⁻¹. The febuxostat (Form VI) is characterized by FT-Raman substantially as shown in FIG. 15 with characteristic peaks at the following wavenumbers: about 1028, about 1317, about 1374, about 1434, about 1512, about 1606, and about 2229 cm⁻¹.

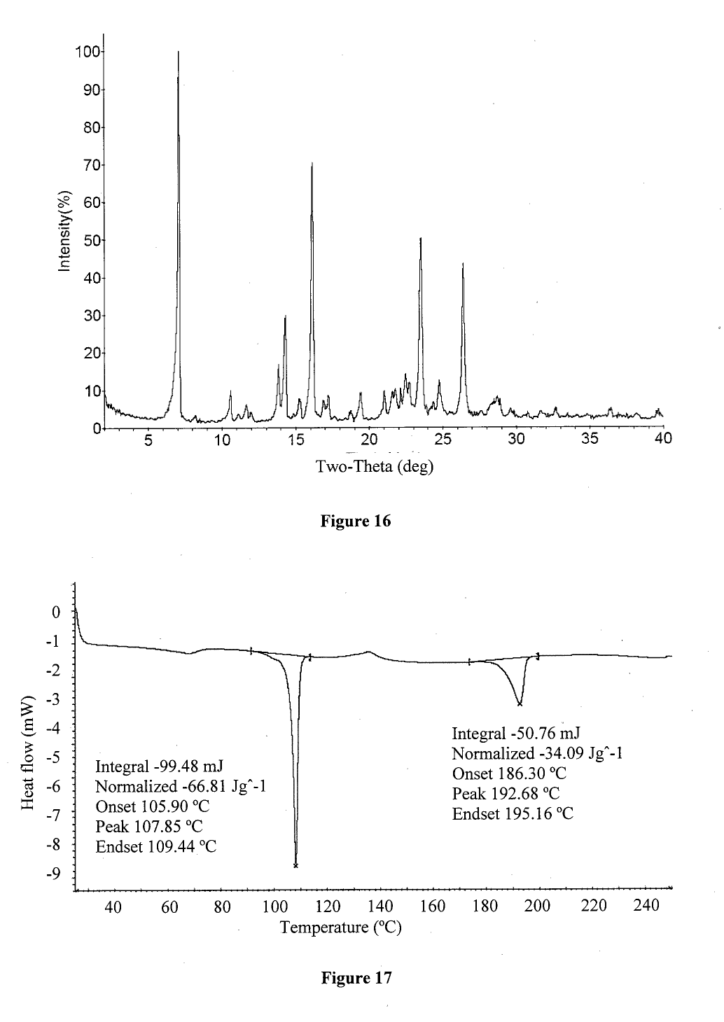

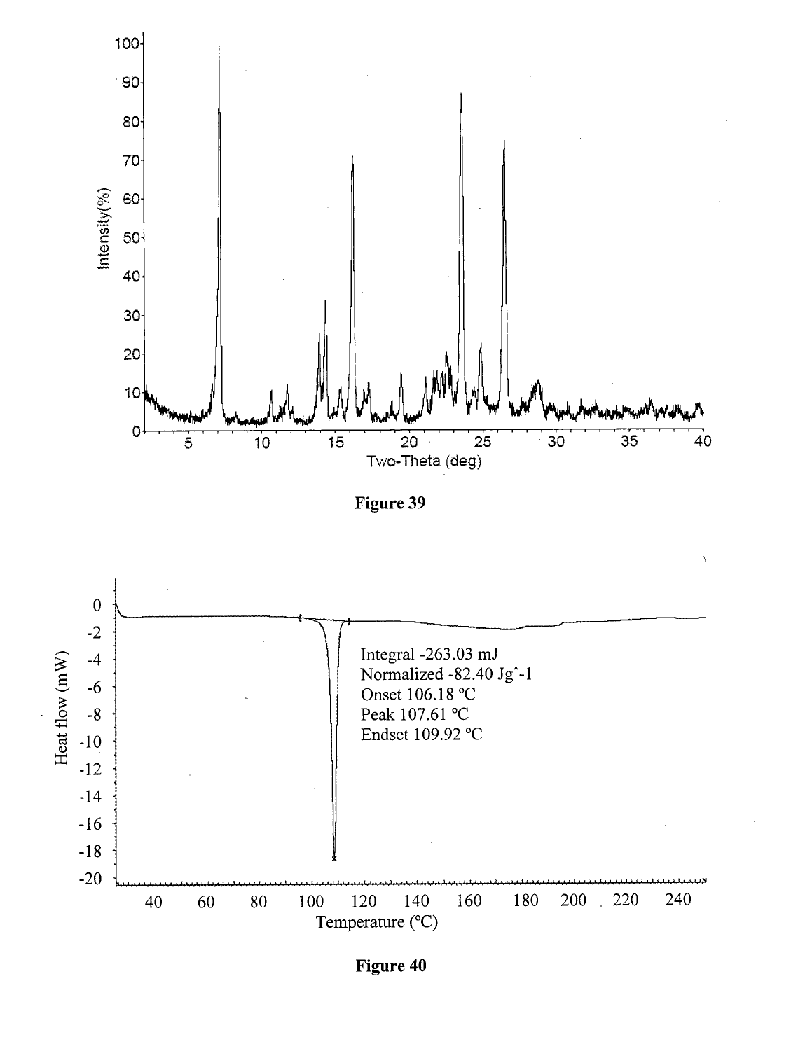

The present invention further provides a crystalline febuxostat DMSO solvate (Form V) which is characterized by an X-ray diffraction pattern substantially as shown in any of FIG. 16 or 39 with peaks at 2 theta values of about 7.1±0.1, 10.6±0.1, 11.7±0.1, 13.8±0.1, 14.3±0.1, 15.2±0.1, 16.2±0.1, 16.9±0.1, 17.2±0.1, 19.4±0.1, 21.0±0.1, 21.6±0.1, 21.8±0.1, 22.1±0.1, 22.5±0.1, 22.7±0.1, 23.5±0.1, 24.8±0.1, 26.4±0.1, and 28.7±0.1. The febuxostat DMSO solvate (Form V) is further characterized using various techniques including infrared absorption, Raman spectrometry, and thermal analysis (e.g. thermogravimetric analysis (TGA) and differential scanning calorimetry (DSC)).

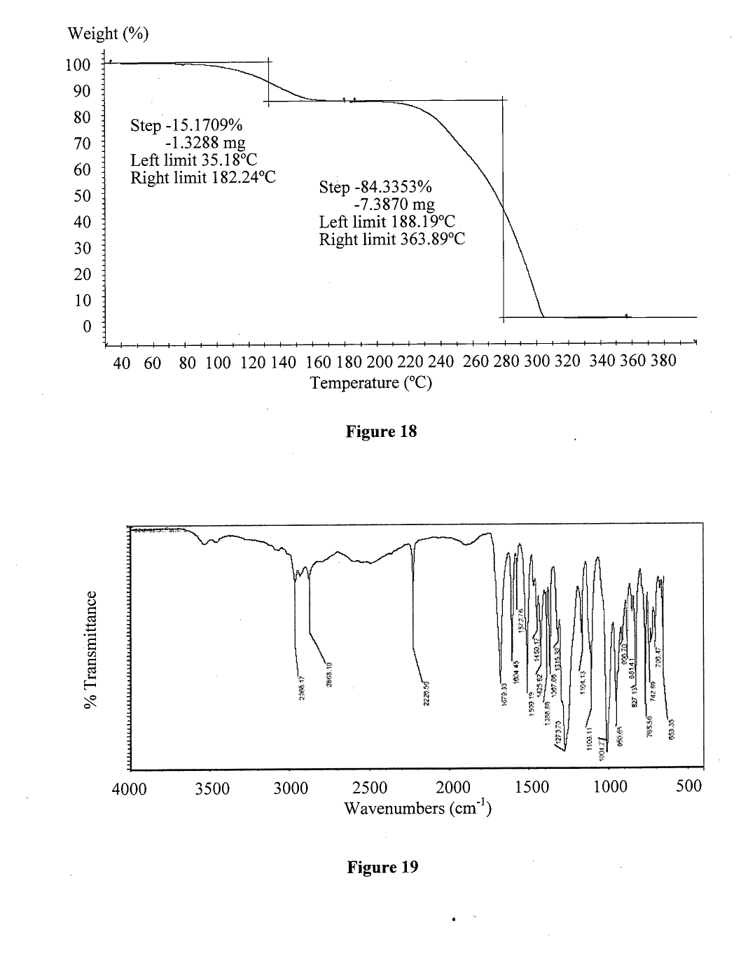

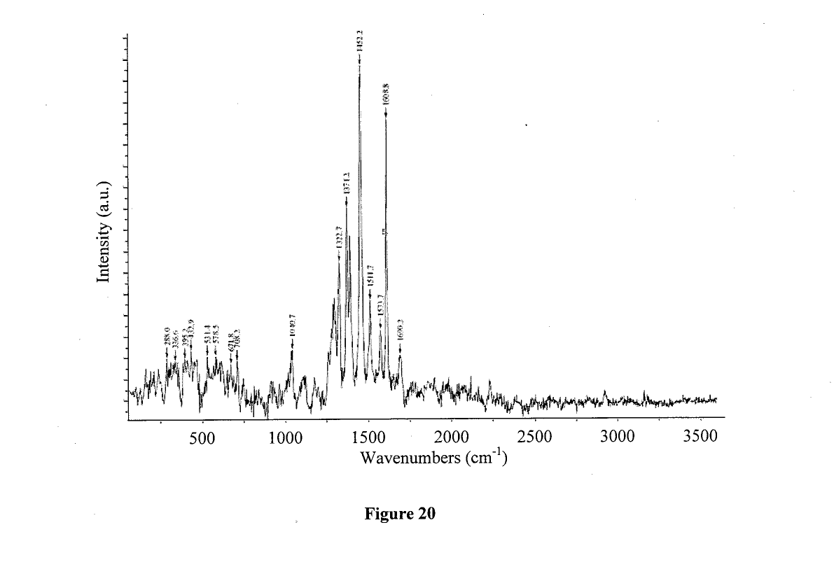

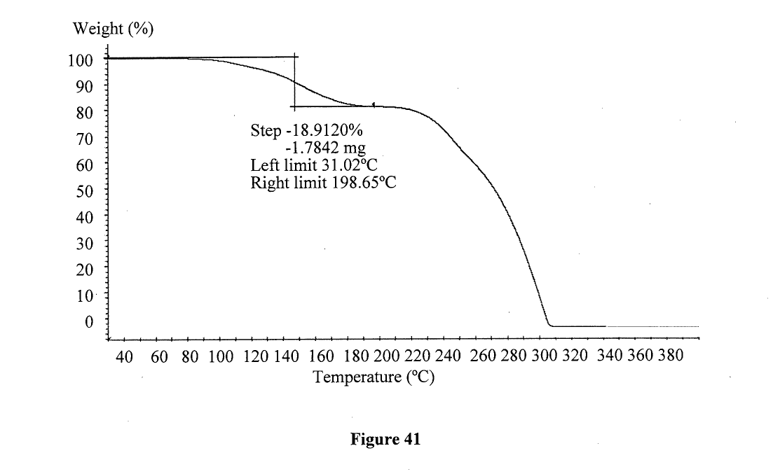

In some embodiments, the febuxostat DMSO solvate (Form V) of the present invention is characterized by a DSC profile substantially as shown in any of FIG. 17 or 40. In other embodiments, the febuxostat (Form V) of the present invention is further characterized by a TGA profile substantially as shown in any of FIG. 18 or 41. The febuxostat (Form V) is further characterized by Infrared spectrum substantially as shown in FIG. 19 with characteristic peaks at the following wavenumbers: about 653, about 706, about 743, about 766, about 827, about 881, about 907, about 951, about 1005, about 1106, about 1164, about 1274, about 1315, about 1368, about 1389, about 1426, about 1450, about 1509, about 1573, about 1604, about 1679, about 2227, about 2868, and about 2966 cm⁻¹. The febuxostat (Form V) is characterized by FT-Raman substantially as shown in FIG. 20 with characteristic peaks at the following wavenumbers: about 288, about 337, about 395, about 433, about 531, about 578, about 672, about 708, about 1041, about 1323, about 1371, about 1452, about 1512, about 1574, about 1609, and about 1690 cm⁻¹.

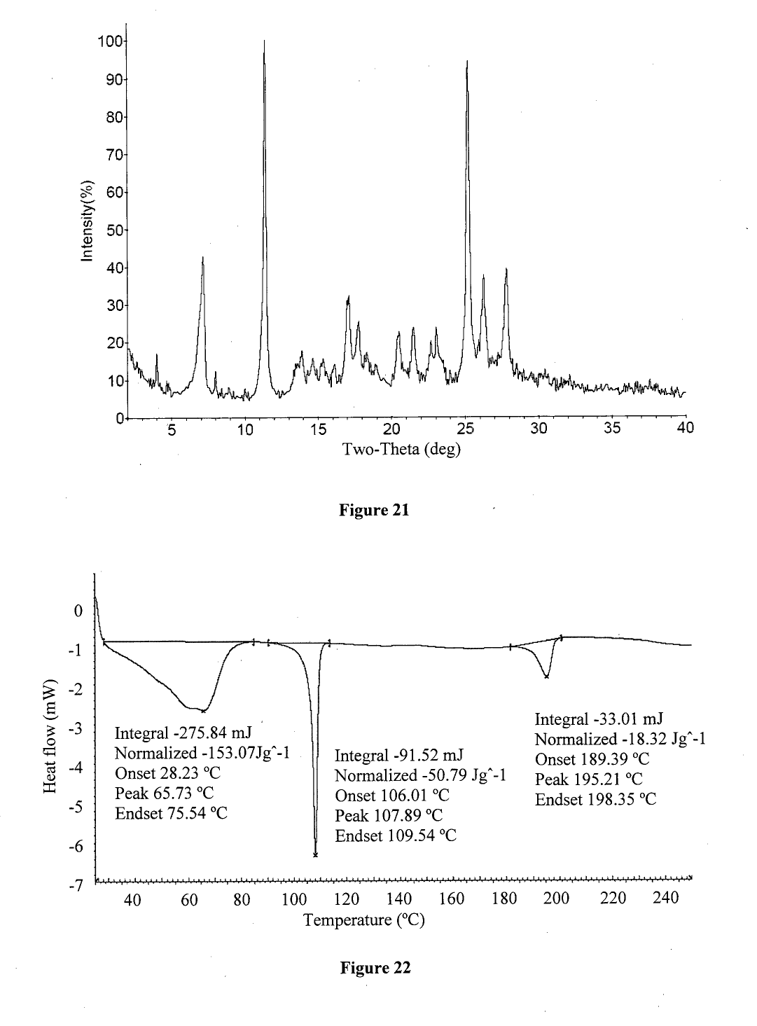

The present invention further provides a crystalline febuxostat DMSO solvate (Form VII) which is characterized by an X-ray diffraction pattern substantially as shown in FIG. 21 with peaks at 2 theta values of about 4.0±0.1, 7.2±0.1, 8.0±0.1, 11.4±0.1, 13.6±0.1, 13.9±0.1, 14.7±0.1, 17.1±0.1, 17.8±0.1, 20.5±0.1, 21.5±0.1, 22.7±0.1, 23.0±0.1, 25.2±0.1, 26.3±0.1, and 27.8±0.1. The febuxostat DMSO solvate (Form VII) is further characterized using various techniques including infrared absorption, Raman spectrometry, and thermal analysis (e.g. thermogravimetric analysis (TGA) and differential scanning calorimetry (DSC)).

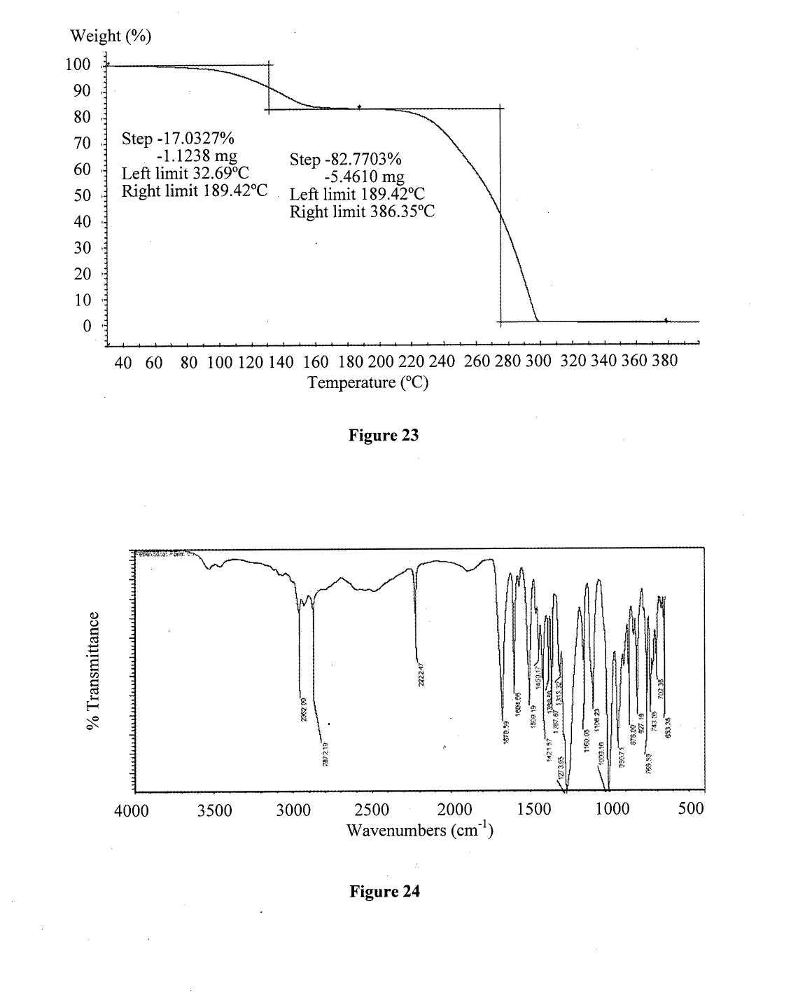

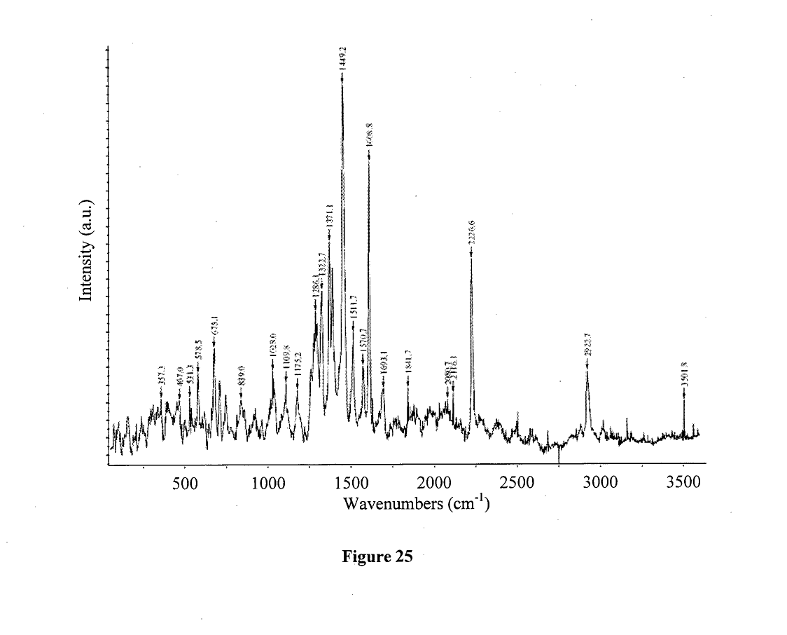

In certain embodiments, the febuxostat DMSO solvate (Form VII) of the present invention is characterized by DSC and TGA profiles substantially as shown in FIGS. 22 and 23, respectively. The febuxostat (Form VII) is further characterized by Infrared spectrum substantially as shown in FIG. 24 with characteristic peaks at the following wavenumbers: about 653, about 702, about 743, about 765, about 827, about 878, about 951, about 1009, about 1106, about 1160, about 1274, about 1315, about 1368, about 1389, about 1422, about 1450, about 1509, about 1605, about 1680, about 2222, about 2872, and about 2962 cm⁻¹. The febuxostat (Form VII) is characterized by FT-Raman substantially as shown in FIG. 25 with characteristic peaks at the following wavenumbers: about 357, about 467, about 531, about 578, about 675, about 839, about 1028, about 1110, about 1175, about 1286, about 1323, about 1371, about 1449, about 1512, about 1571, about 1609, about 1693, about 1842, about 2081, about 2116, about 2227, about 2923, and about 3502 cm⁻¹.

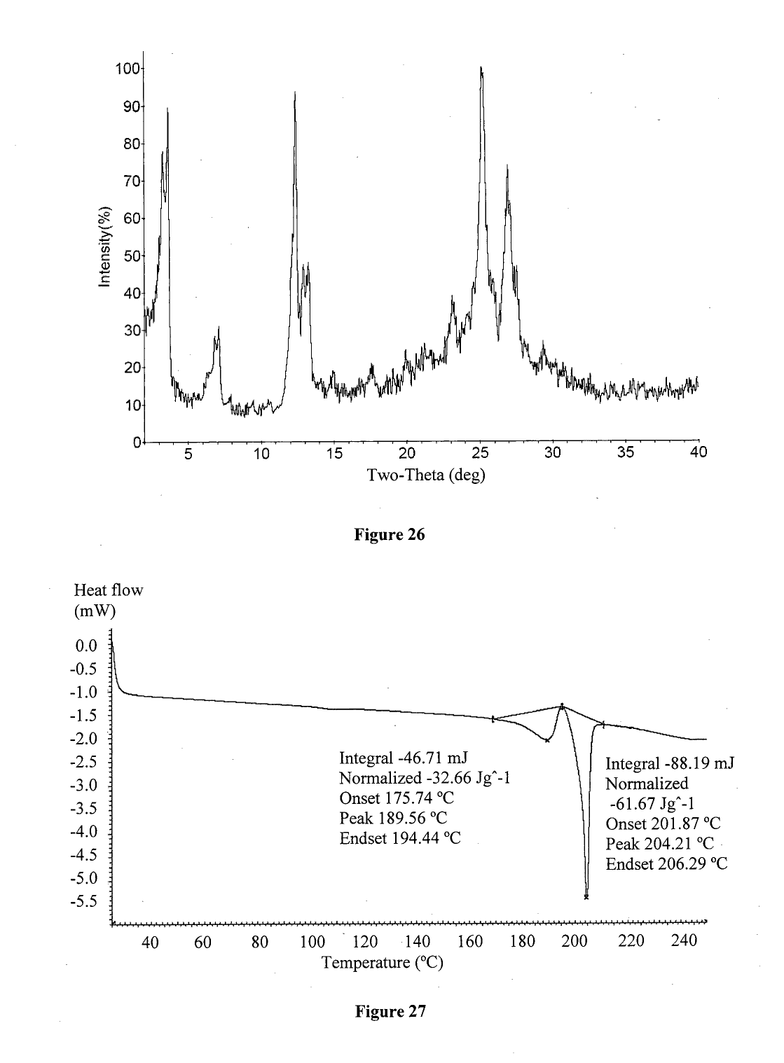

Provided herein is an anhydrous form of febuxostat (Form VIII) which is characterized by an X-ray diffraction pattern substantially as shown in FIG. 26 with peaks at 2 theta values of about 3.6±0.1, 7.1±0.1, 12.4±0.1, 13.3±0.1, 17.6±0.1, 23.1±0.1, 25.2±0.1, 27.0±0.1, and 27.6±0.1. The anhydrous febuxostat (Form VIII) is further characterized using various techniques including infrared absorption, Raman spectrometry, and thermal analysis (e.g. thermogravimetric analysis (TGA) and differential scanning calorimetry (DSC)).

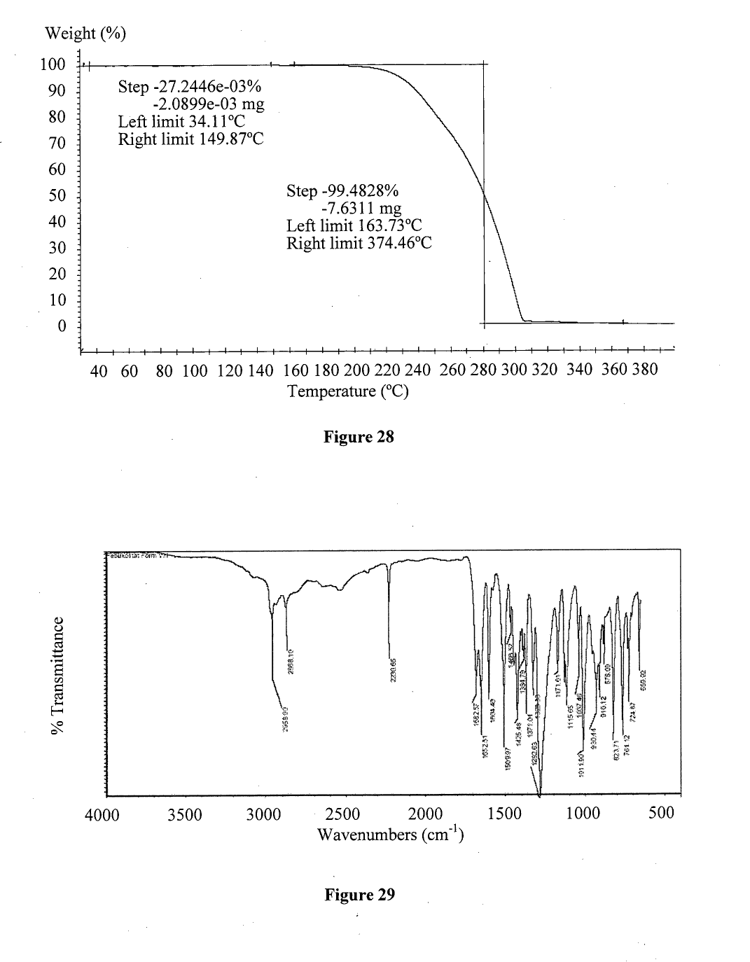

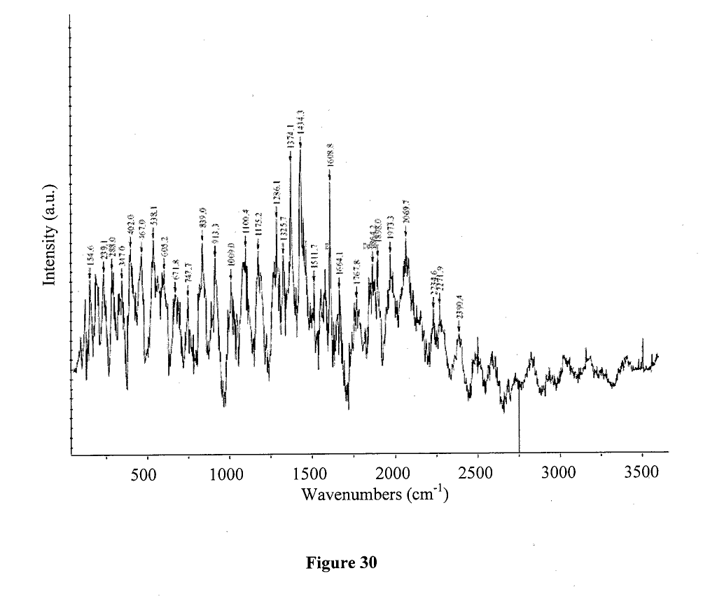

In various embodiments, the anhydrous febuxostat (Form VIII) of the present invention is characterized by DSC and TGA profiles substantially as shown in FIGS. 27 and 28, respectively. The anhydrous febuxostat (Form VIII) is further characterized by Infrared spectrum substantially as shown in FIG. 29 with characteristic peaks at the following wavenumbers: about 660, about 725, about 764, about 824, about 878, about 910, about 930, about 1012, about 1037, about 1116, about 1172, about 1283, about 1328, about 1371, about 1385, about 1425, about 1467, about 1510, about 1604, about 1653, about 1683, about 2231, about 2868, and about 2958 cm⁻¹. The anhydrous febuxostat (Form VIII) is characterized by FT-Raman substantially as shown in FIG. 30 with characteristic peaks at the following wavenumbers: about 155, about 239, about 288, about 347, about 402, about 467, about 538, about 605, about 672, about 748, about 839, about 913, about 1009, about 1100, about 1175, about 1286, about 1326, about 1374, about 1434, about 1512, about 1609, about 1664, about 1768, about 1864, about 1898, about 1973, about 2070, about 2235, about 2272, about 2390 cm⁻¹.

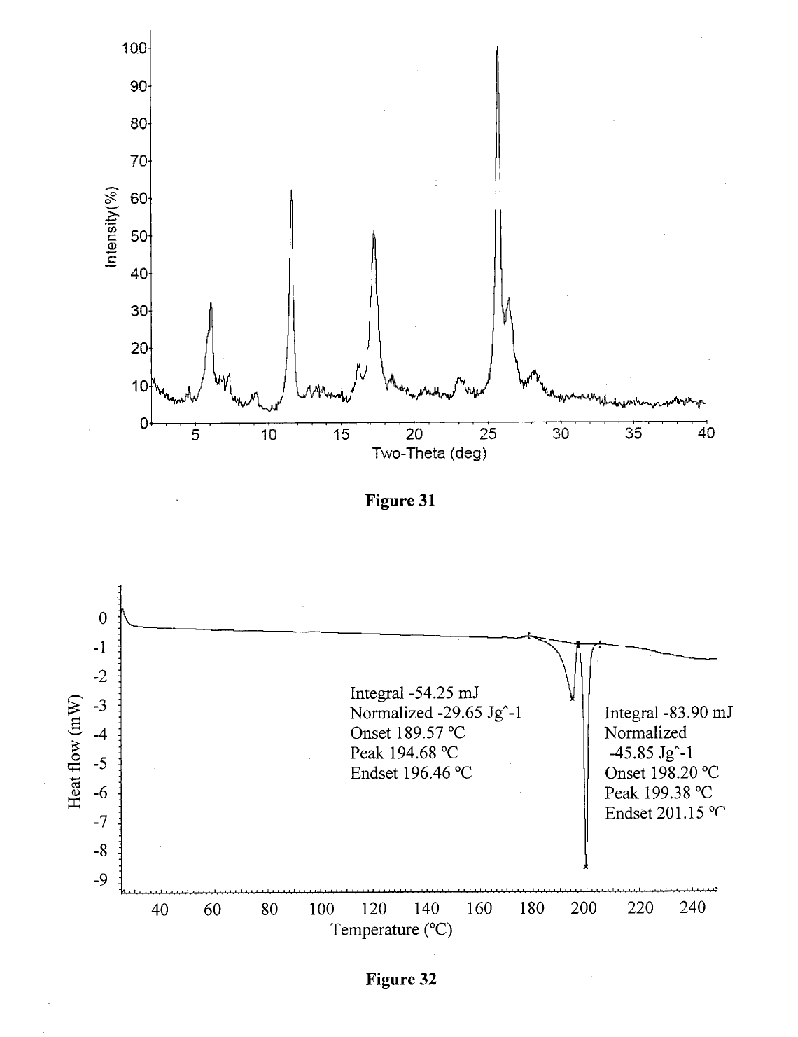

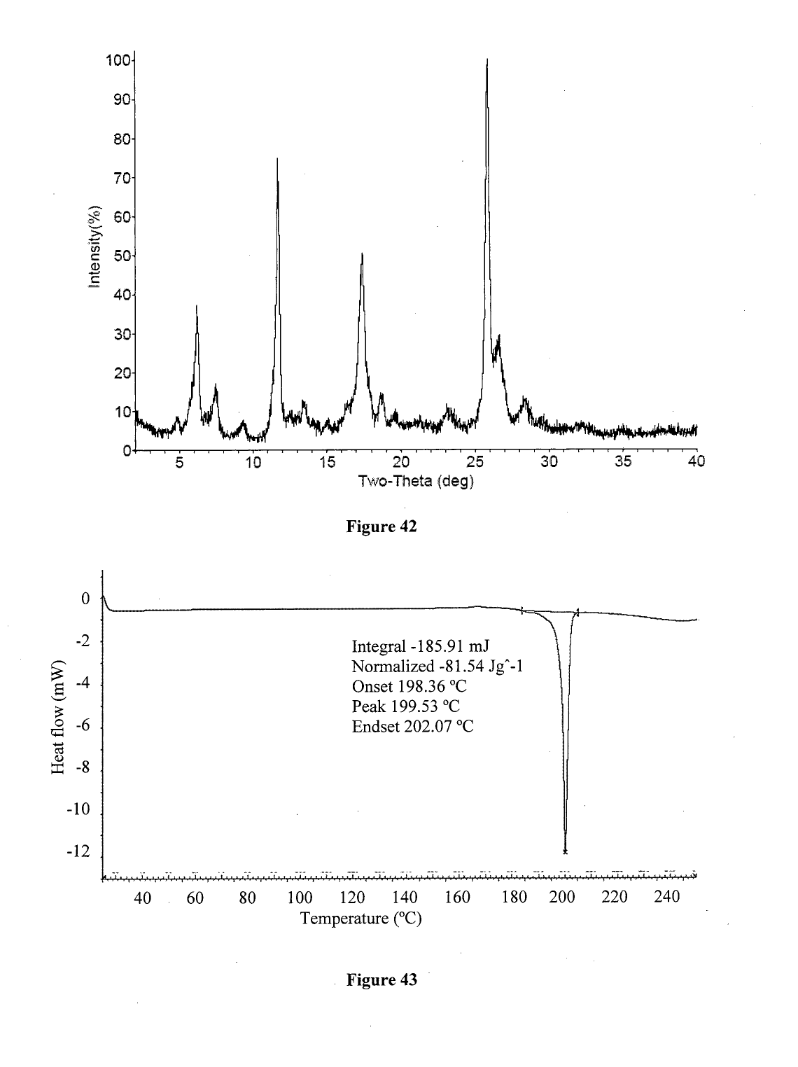

The present invention further provides an anhydrous form of febuxostat (Form IX) which is characterized by an X-ray diffraction pattern substantially as shown in any of FIG. 31 or 42 with peaks at 2 theta values of about 4.6±0.1, 6.1±0.1, 7.3±0.1, 9.2±0.1, 11.6±0.1, 13.3±0.1, 16.3±0.1, 17.3±0.1, 18.5±0.1, 23.0±0.1, 25.7±0.1, 26.5±0.1 and 28.3±0.1. The anhydrous febuxostat (Form IX) is further characterized using various techniques including infrared absorption, Raman spectrometry, and thermal analysis (e.g. thermogravimetric analysis (TGA) and differential scanning calorimetry (DSC)).

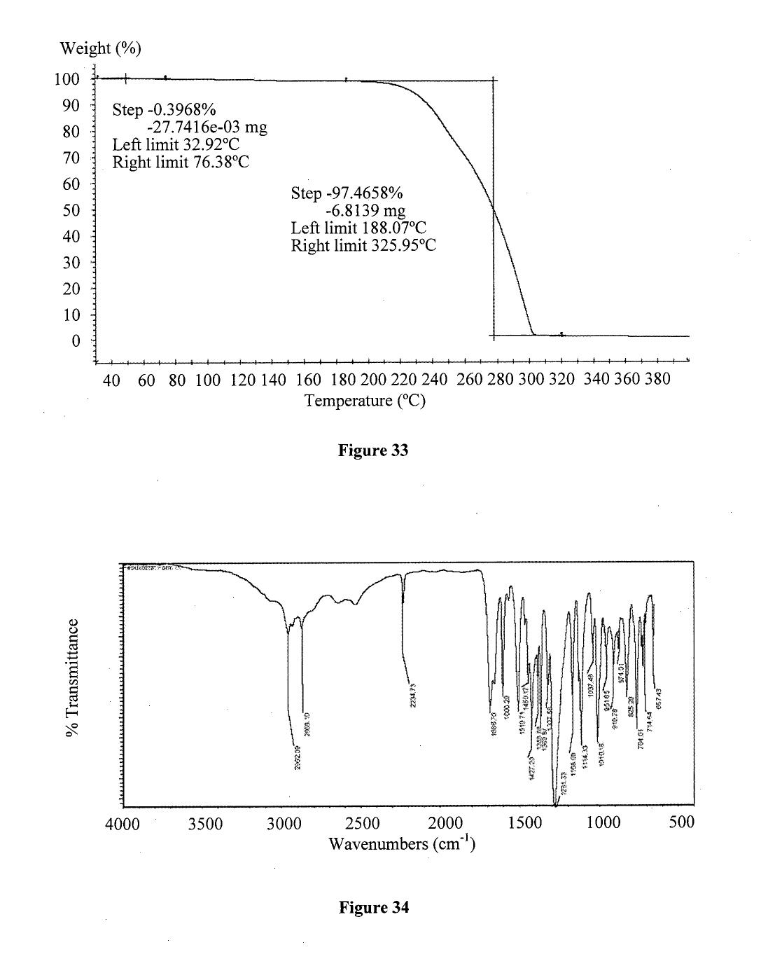

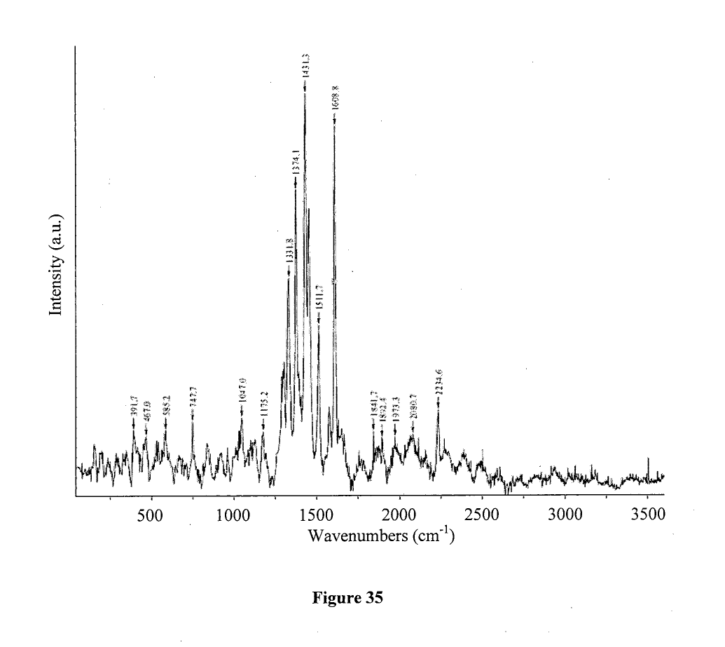

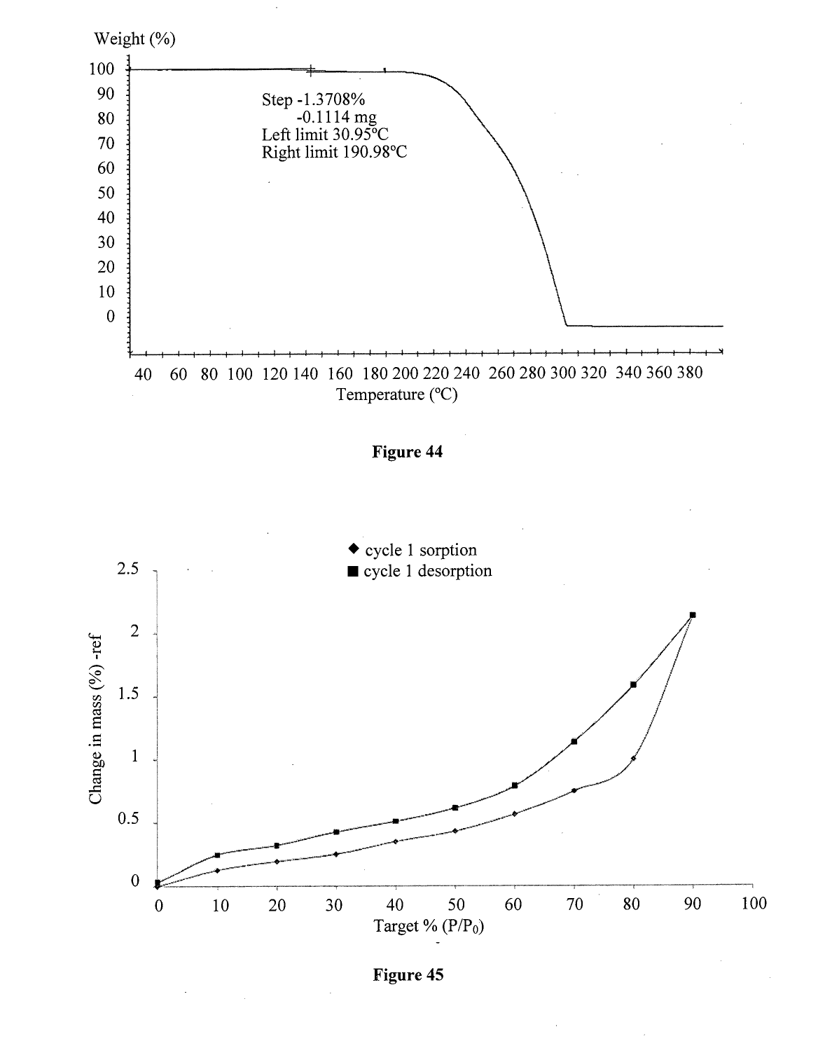

In certain embodiments, the anhydrous febuxostat (Form IX) of the present invention is characterized by a DSC profile substantially as shown in any of FIG. 32 or 43. In other embodiments, the anhydrous febuxostat (Form IX) of the present invention is characterized by a TGA profile substantially as shown in any of FIG. 33 or 44. The anhydrous febuxostat (Form IX) is further characterized by Infrared spectrum substantially as shown in FIG. 34 with characteristic peaks at the following wavenumbers: about 657, about 715, about 764, about 825, about 874, about 911, about 952, about 1010, about 1037, about 1114, about 1168, about 1281, about 1328, about 1370, about 1389, about 1427, about 1450, about 1511, about 1606, about 1687, about 2235, about 2868 and about 2962 cm⁻¹. The anhydrous febuxostat (Form IX) is characterized by FT-Raman substantially as shown in FIG. 35 with characteristic peaks at the following wavenumbers: about 392, about 467, about 585, about 748, about 1047, about 1175, about 1332, about 1374, about 1431, about 1512, about 1609, about 1842, about 1892, about 1973, about 2081, and about 2235 cm⁻¹.

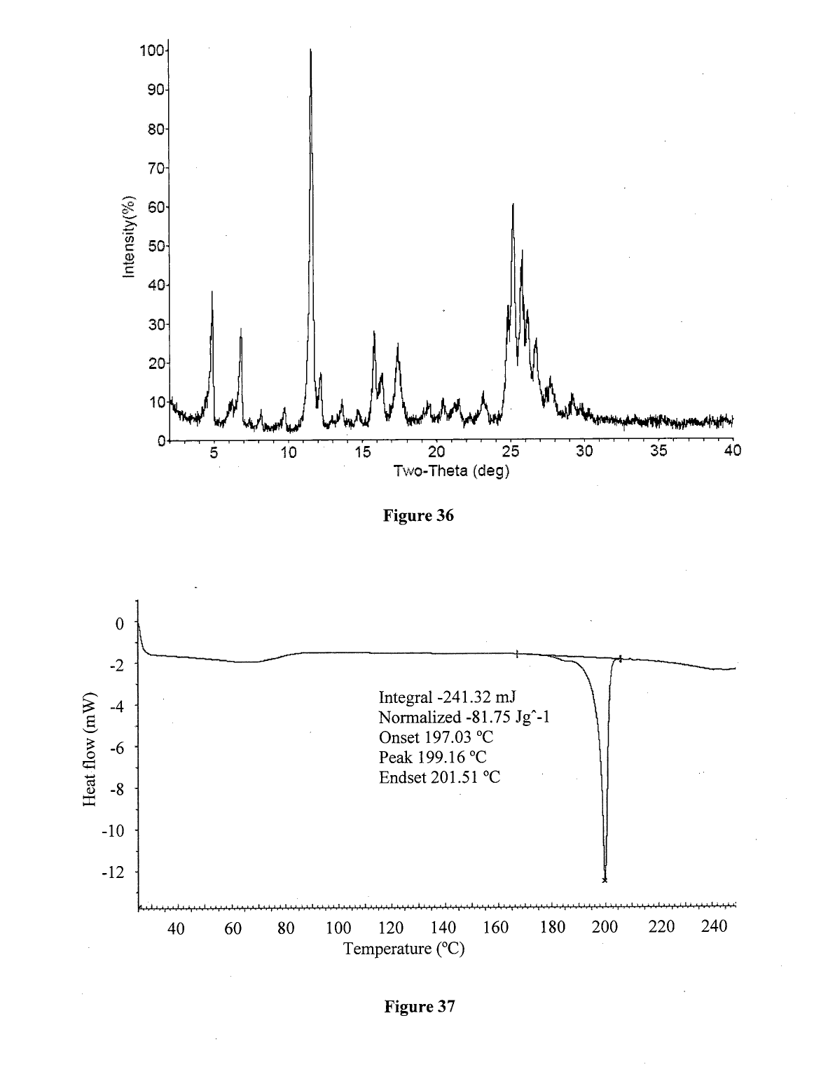

The present invention further provides a crystalline febuxostat hydrate (Form XI) which is characterized by an X-ray diffraction pattern substantially as shown in FIG. 36 with peaks at 2 theta values of about 4.9±0.1, 6.2±0.1, 6.8±0.1, 8.2±0.1, 9.7±0.1, 11.6±0.1, 12.2±0.1, 13.6±0.1, 15.8±0.1, 16.3±0.1, 17.5±0.1, 19.4±0.1, 20.5±0.1, 21.3±0.1, 21.5±0.1, 23.2±0.1, 24.8±0.1, 25.2±0.1, 25.8±0.1, 26.2±0.1, 26.8±0.1, 27.8±0.1, 29.2±0.1 and 29.8±0.1. The febuxostat hydrate (Form XI) is further characterized using various techniques including thermal analysis (e.g. thermogravimetric analysis (TGA) and differential scanning calorimetry (DSC)).

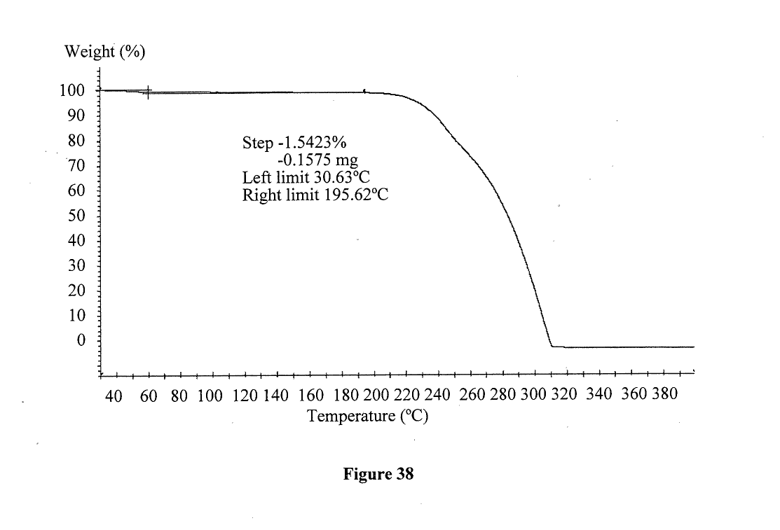

In various embodiments, the febuxostat hydrate (Form XI) of the present invention is characterized by a DSC profile substantially as shown in FIG. 37, with an endothermic peak at about 199° C. The febuxostat hydrate (Form XI) is further characterized by a TGA profile substantially as shown in FIG. 38 with a weight loss of about 1.5% from about 31° C. to about 196° C.

The present invention further provides processes for the preparation of the febuxostat forms of the present invention. The processes include thermal precipitations and precipitations from supersaturated solutions. In particular, these processes involve the use of febuxostat, for example febuxostat API as the starting material or any other commercially available febuxostat or febuxostat prepared by any methods known in the art, including, for example, the methods described in EP 0513379, JP 1993500083, U.S. Pat. No. 5,614,520 and WO 92/09279, JP 10-045733, JP10-139770, JP 1994345724 (JP 6-345724), in publications in Heterocycles, 1998, 47: 857-864 and Org. Lett., 2009, 11(8): 1733-1736 and in PCT international patent application PCT/IL2010/000807. The contents of the aforementioned references are incorporated by reference herein. According to one embodiment, the febuxostat starting material is heated until a melt is obtained, preferably under vacuum followed by controlled precipitation by slow/fast cooling. According to another embodiment, the febuxostat starting material is dissolved in a suitable solvent or a mixture of solvents to prepare saturated solutions at room temperatures or at temperatures below the solvent boiling point. The solvent is then removed by evaporation. In additional embodiments, the febuxostat starting material is dissolved in one solvent followed by the addition of an anti-solvent to afford the precipitation of a febuxostat form of the present invention. In further embodiments, the febuxostat starting material is dissolved in a solvent or a mixture of solvents while heated. The hot solution is then cooled to afford the precipitation of a febuxostat form of the present invention.

Additional methods for the preparation of the febuxostat forms of the present invention include, for example, precipitation from a suitable solvent, precipitation by cooling under vacuum, sublimation, growth from a melt, solid state transformation from another phase, precipitation from a supercritical fluid, and jet spraying. Techniques for precipitation from a solvent or solvent mixture include, for example, evaporation of the solvent, decreasing the temperature of the solvent mixture, freeze-drying the solvent mixture, and addition of anti-solvents (counter-solvents) to the solvent mixture. The term “anti-solvent” as used herein refers to a solvent in which the compound has low solubility.

Suitable solvents and anti-solvents for preparing the forms of the present invention include polar and non-polar solvents. The choice of solvent or solvents is typically dependent upon one or more factors, including the solubility of the compound in such solvent and vapor pressure of the solvent. Combinations of solvents may be employed; for example, the compound may be solubilized into a first solvent followed by the addition of an anti-solvent to decrease the solubility of the compound in the solution and to induce precipitation. Suitable solvents include, but are not limited to, polar aprotic solvents, polar protic solvents, and mixtures thereof. Particular examples of suitable polar protic solvents include, but are not limited to, alcohols such as methanol (MeOH), ethanol (EtOH), 1-butanol, and isopropanol (IPA). Particular examples of suitable polar aprotic solvents include, but are not limited to, acetonitrile (ACN), tetrahydrofuran (THF), 2-methyltetrahydrofuran (2MeTHF), N-methyl-2-pyrrolidone (NMP), dichloromethane, acetone, dimethylformamide (DMF), and dimethylsulfoxide (DMSO). Each possibility represents a separate embodiment of the present invention.

The febuxostat forms of the present invention may be obtained by distillation or solvent addition techniques such as those known to those skilled in the art. Suitable solvents for this purpose include any of those solvents described herein, including protic polar solvents, such as alcohols (including those listed above), aprotic polar solvents (including those listed above), ketones (for example, acetone, methyl ethyl ketone (MEK), and methyl isobutyl ketone) and also esters (ethyl acetate (EtOAc)). Each possibility represents a separate embodiment of the present invention.

Exemplary processes used to prepare each of the febuxostat forms of the present invention are provided herein.

Methods for “precipitation from solution” include, but are not limited to, evaporation of a solvent or solvent mixture, a concentration method, a slow cooling method, a fast cooling method, a reaction method (diffusion method, electrolysis method), a hydrothermal growth method, a fusing agent method, and so forth. The solution can be a saturated solution or a supersaturated solution, optionally heated to temperatures below the solvent boiling point. The recovery of the forms can be done for example, by filtering the suspension and drying. Alternatively, the solvents may be removed by rotary evaporation at desired temperatures.

The febuxostat forms of the present invention can be prepared using fast/slow precipitation from saturated solutions in different solvents or mixture of solvents which are allowed to evaporate, preferably at room temperatures. The obtained precipitate may further by washed with a suitable solvent (e.g. ACN). In additional embodiments, the obtained precipitate may further be dried at room temperatures or at temperatures below the solvent boiling point (e.g. 40° C.), preferably under vacuum. Alternatively, the saturated solutions can be heated followed by their cooling to induce precipitation as is known in the art.

The febuxostat forms of the present invention can be prepared using solvent/anti-solvent systems. Typically the active ingredient is dissolved in a suitable solvent, optionally at temperatures below the solvent boiling point. An anti-solvent is then added to induce precipitation of the desired form.

The febuxostat forms of the present invention can be prepared by melting the active ingredient, preferably in an inert atmosphere. The melt is then cooled to afford precipitation of the desired form.

The febuxostat forms of the present invention can be prepared by the slurry method as is well known in the art. Suspensions of the active ingredient in different solvents or mixture of solvents are prepared and shaken for long intervals (typically 24 hours).

Within the scope of the present invention are high pressure techniques where the active ingredient is compressed using various forces (e.g. grinding) as is known in the art.

As contemplated herein, the febuxostat forms of the present invention can further be obtained using lyophilization wherein the compound is dissolved in water, followed by a freeze-drying procedure.

The novel forms of the present invention are useful as pharmaceuticals for treating hyperuricaemia. The present invention thus provides pharmaceutical compositions comprising any of the febuxostat forms disclosed herein and a pharmaceutically acceptable carrier. The forms of the present invention can be safely administered orally or non-orally. Routes of administration include, but are not limited to, oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual. Each possibility represents a separate embodiment of the invention. Typically, the febuxostat forms of the invention are administered orally. The pharmaceutical compositions can be formulated as tablets (including e.g. film-coated tablets), powders, granules, capsules (including soft capsules), orally disintegrating tablets, and sustained-release preparations as is well known in the art. Each possibility represents a separate embodiment of the invention.

Pharmacologically acceptable carriers that may be used in the context of the present invention include various organic or inorganic carriers including, but not limited to, excipients, lubricants, binders, disintegrants, water-soluble polymers and basic inorganic salts. The pharmaceutical compositions of the present invention may further include additives such as, but not limited to, preservatives, antioxidants, coloring agents, sweetening agents, souring agents, bubbling agents and flavorings.

Suitable excipients include e.g. lactose, D-mannitol, starch, cornstarch, crystalline cellulose, light silicic anhydride and titanium oxide. Suitable lubricants include e.g. magnesium stearate, sucrose fatty acid esters, polyethylene glycol, talc and stearic acid. Suitable binders include e.g. hydroxypropyl cellulose, hydroxypropylmethyl cellulose, crystalline cellulose, a-starch, polyvinylpyrrolidone, gum arabic powder, gelatin, pullulan and low-substitutional hydroxypropyl cellulose. Suitable disintegrants include e.g. crosslinked povidone (any crosslinked 1-ethenyl-2-pyrrolidinone homopolymer including polyvinylpyrrolidone (PVPP) and 1-vinyl-2-pyrrolidinone homopolymer), crosslinked carmellose sodium, carmellose calcium, carboxymethyl starch sodium, low-substituted hydroxypropyl cellulose, cornstarch and the like. Suitable water-soluble polymers include e.g. cellulose derivatives such as hydroxypropyl cellulose, polyvinylpyrrolidone, hydroxypropylmethyl cellulose, methyl cellulose and carboxymethyl cellulose sodium, sodium polyacrylate, polyvinyl alcohol, sodium alginate, guar gum and the like. Suitable basic inorganic salts include e.g. basic inorganic salts of sodium, potassium, magnesium and/or calcium. Particular embodiments include the basic inorganic salts of magnesium and/or calcium. Basic inorganic salts of sodium include, for example, sodium carbonate, sodium hydrogen carbonate, disodiumhydrogenphosphate, etc. Basic inorganic salts of potassium include, for example, potassium carbonate, potassium hydrogen carbonate, etc. Basic inorganic salts of magnesium include, for example, heavy magnesium carbonate, magnesium carbonate, magnesium oxide, magnesium hydroxide, magnesium metasilicate aluminate, magnesium silicate, magnesium aluminate, synthetic hydrotalcite, aluminahydroxidemagnesium and the like. Basic inorganic salts of calcium include, for example, precipitated calcium carbonate, calcium hydroxide, etc.

Suitable preservatives include e.g. sodium benzoate, benzoic acid, and sorbic acid. Suitable antioxidants include e.g. sulfites, ascorbic acid and a-tocopherol. Suitable coloring agents include e.g. food colors such as Food Color Yellow No. 5, Food Color Red No. 2 and Food Color Blue No. 2 and the like. Suitable sweetening agents include e.g. dipotassium glycyrrhetinate, aspartame, stevia and thaumatin. Suitable souring agents include e.g. citric acid (citric anhydride), tartaric acid and malic acid. Suitable bubbling agents include e.g. sodium bicarbonate. Suitable flavorings include synthetic substances or naturally occurring substances, including e.g. lemon, lime, orange, menthol and strawberry.

In some embodiments, the present invention provides a pharmaceutical composition comprising as an active ingredient a single crystalline form of febuxostat of the present invention (e.g. the anhydrous febuxostat (Form IX) or any one of forms IV, VI, VII or VIII) and a pharmaceutically acceptable carrier. In certain embodiments, the pharmaceutically acceptable carrier comprises an excipient such as lactose, crystalline cellulose and starch, a binder such as hydroxypropyl cellulose, coating such as polyethylene glycol, a disintegrant such as carmellose, hydroxypropyl cellulose and crosspovidone and other known binders, lubricants, coating agents, plasticizers, diluents, colorants, and preservatives as defined hereinabove.

The febuxostat forms of the present invention are particularly suitable for oral administration in the form of tablets, capsules, pills, dragées, powders, granules and the like. A tablet may be made by compression or molding, optionally with one or more excipients as is known in the art. Specifically, molded tablets may be made by molding in a suitable machine a mixture of the powdered active ingredient moistened with an inert liquid diluent.

The tablets and other solid dosage forms of the pharmaceutical compositions described herein may optionally be scored or prepared with coatings and shells, such as enteric coatings and other coatings well known in the art. They may also be formulated so as to provide slow or controlled release of the active ingredient therein using, for example, hydroxypropylmethyl cellulose in varying proportions to provide the desired release profile, other polymer matrices and the like. The active ingredient can also be in micro-encapsulated form, if appropriate, with one or more of the above-described excipients.

The present invention provides a method of treating hyperuricaemia comprising administering to a subject in need thereof an effective amount of a composition comprising any one of the febuxostat forms of the present invention.

“A therapeutically effective amount” as used herein refers to an amount of an agent which is effective, upon single or multiple dose administration to the subject in providing a therapeutic benefit to the subject. In additional embodiments, the febuxostat forms of the present invention are used for the preparation of a medicament for treating hyperuricaemia.

The present invention further provides the administration of the febuxostat forms in combination therapy with one or more other active ingredients. The combination therapy may include the two or more active ingredients within a single pharmaceutical composition as well as the two or more active ingredients in two separate pharmaceutical compositions administered to the same subject simultaneously or at a time interval determined by a skilled artisan.

The principles of the present invention are demonstrated by means of the following non-limiting examples.

### EXAMPLES

### Example 1

### General Preparation Methods of Febuxostat Polymorphs

1. Reagents

Febuxostat API was manufactured according to the teachings of Hasegawa in Heterocycles 47, 857-64, 1998.

Acetic acid, AR, Jiangsu Qiangsheng chemical reagents, Lot No. 20100221

Acetonitrile, HPLC grade, Sigma, Lot No. 07278PH or Merck, Lot No. SB0SF60081

Ammonium hydrogen carbonate, AR, Shanghai Shisihewei chemical, Lot No. 1007101

Ethanol, HPLC grade, Sigma, Lot No. 11085CH

DMF, HPLC grade, Merck, Lot No. SB0S600093

DMSO, HPLC grade, Sigma, Lot No. 05737BH or 27496kk

Hydrochloric acid, AR, SCRC, Lot No. T20100302

Methanol, AR, SCRC, Lot No. T20090912 or HPLC grade, Merck, Lot No. SB0SF60085

Ethanol Acetate, AR, Yixing Secondary Chemical Company, Lot No. 090607 or SCRC, Lot No. T20100126

Isopropyl alcohol, AR, Sinopharm Chemical Reagent Co. Ltd, Lot No. T20090813

Acetone, AR, Sinopharm Chemical Reagent Co. Ltd, Lot No. 090104

THF, AR, Yixing Secondary Chemical, Lot No. 090901 or HPLC grade, Merck, Lot No. IL8IF58153

1-Butanol, AR, SCRC, Lot No. T20080818

Lecithin, laboratory grade, Fisher chemical, Lot No. 091043

MEK, AR, SCRC, Lot No. T20090724

2-Me-THF, AR, Shanghai Jiachen Chemical Reagent Co. Ltd, Lot No. 090323

N-methylpyrrolidone, HPLC grade, Sigma-Aldrich, Lot No. S86863-279

Monobasic potassium phosphate, AR, SCRC, Lot No. F20100413

Potassium biphthalate, GR, Shanghai experimental reagents, Lot No. 070423

Potassium chloride, AR, SCRC, Lot No. F20090409

Sodium dihydrogen phosphate, AR, SCRC, Lot No. F20100330

Sodium hydroxide, AR, Shanghai Lingfeng chemical reagents, Lot No. 081118

Sodium lauryl sulfate, AR, SCRC, Lot No. F20080521

Sodium taurocholate, laboratory grade, Sigma, Lot No. 0001428479

Sodium chloride, AR, Jiangsu Qiangsheng chemical reagents, Lot No. 20100112

2. Instruments

SMS DVS

Agilent 1200 HPLC

Mettler Toledo Seven Multi pH meter

Binder KBF115 Stability Chamber

GZX-9140 MBE oven

ERWEKA SVM203 Tapped Density Tester

Sartorius CP 225D Balance

Mettler Toledo MX5 Balance

ELGA Water Purification Equipment

Mettler Toledo DSC 1

Mettler Toledo TGA/DSC 1

Rigaku D/MAX 2200 X-ray powder diffractometer

Thermo Nicolet 380 FT-IR

NMR Varian 400

Nikon LV100 Polarized Light Microscopy

Boxun vacuum oven DZF-6050

Eyela FDU-1100 freeze dryer

Jobin Yvon LabRam-1B FT-Raman

3. XRPD, DSC, TGA, FTIR, FT-Raman and HPLC Methods

3.1 XRPD Method

Details of XRPD method used in the tests are mentioned below:
X-ray Generator: Cu, kα, (λ=1.54056 Å)
Tube Voltage: 40 kV, Tube Current: 40 mA
DivSlit: 1 deg
DivH.L.Slit: 10 mm
SctSlit: 1 deg
RecSlit: 0.15 mm
Monochromator: Fixed Monochromator
Scanning Scope: 2-40 deg
Scanning Step: 10 deg/min

3.2 DSC and TGA Methods

Details of DSC method used in the tests are mentioned below:
Heat from 25° C. to 250° C. at 10° C./min

Details of DSC (topem) method used in the tests are mentioned below:
Heat from 0° C. to 250° C. at 2° C./min

Details of TGA method used in the tests are mentioned below:

Heat from 30° C. to 400° C. at 10° C./min

3.3 FT-IR and FT-Raman Method

Details of FT-IR method used in the tests are mentioned below:
No. of scan: 32
Time for collection: 38 s
Scan Range: 400-4000 cm⁻¹
Resolution: 4

Details of FT-Raman method used in the tests are mentioned below:
Laser wave: 632.8 nm
Power: 1 mW
Resolution: 1 cm⁻¹
Time for integration: 50 s

3.4 HPLC Method

Details of HPLC method used in the tests are mentioned below:

The detailed chromatographic conditions are listed hereinbelow. The typical retention time of febuxostat main peak is 8.2 min.

| Instrument | Agilent 1200 HPLC | | |
|---|---|---|---|
| Column | Symmetry shield-RP18, 4.6 mm * 150 mm, 3.5 μm | | |
| Mobile phase | A: 20 mM NH₄HCO₃ in water, pH 7.0 adjusted | | |
| | with acetic acid | | |
| | B: ACN, v/v | | |
| Gradient | Time (min) | A % | B % |
| | Initial | 98 | 2 |
| | 15.00 | 2 | 98 |
| | 20.00 | 2 | 98 |
| | 20.10 | 98 | 2 |

| | |
|---|---|
| Runtime | 20 min |
| Post run time | 5 min |
| Column temperature | 25° C. |
| Injection Volume | 10 μL |
| Mobile rate | 1.0 mL/min |

4. General Preparation Methods

4.1 Method 1: Slow Precipitation from Saturated Solutions

Solutions of Febuxostat API (lot number CCS-1058/B361/B-IV/06) in different solvents were prepared and filtered through 0.22 μm filter into clean vessels. Solvents were evaporated at room temperatures to form crystals. Febuxostat hydrate (Form II) was identified by this method, as set forth in the Examples below.

4.2 Method 2: Solvent-Thermal Heating/Cooling

Saturated solutions of Febuxostat API (lot number CCS-1058/B361/B-IV/06) in different solvents or mixture of solvents were prepared at 50° C. The solutions were then cooled down at 5° C. to form crystals. Febuxostat Form IV (NMP solvate) and febuxostat Form V (DMSO solvate) were identified by this method, as set forth in the Examples below.

4.3 Method 3: Anti-Solvent Precipitation

Saturated solutions of Febuxostat API (lot number CCS-1058/B361/B-IV/06) with different solvents were prepared at 25/50° C. An anti-solvent (kept at 25° C.) was then added to precipitate out crystals. Febuxostat Form VI (NMP solvate) and febuxostat Form VII (DMSO solvate) were identified by this method, as set forth in the Examples below.

4.4 Method 4: Thermal Heating/Cooling

Febuxostat API (lot number CCS-1058/B361/B-IV/06) was heated to melt under vacuum. The melted compound was then rapidly or slowly cooled. Anhydrous febuxostat Form VIII was identified by this method, as set forth in the Examples below.

4.5 Method 5: Fast Precipitation from Saturated Solutions

Saturated solutions of febuxostat API (lot number CCS-1058/B361/B-IV/06) were prepared with different solvents at room temperatures. The solvents were then removed by rotary evaporator below 50° C. Anhydrous febuxostat Form IX was identified by this method, as set forth in the Examples below.

4.6 Method 6: Slow Precipitation from Saturated Solutions Followed by Drying

A clear solution of febuxostat API (lot number CCS-1058/B361/B-IV/06) in THF was prepared. The THF was then evaporated in fume hood at room temperature and the residual solid was further dried in a vacuum oven at 40° C. overnight. Febuxostat hydrate (Form XI) was identified by this method, as set forth in the Examples below.

5. General Assessment Methods

5.1 Hygroscopicity Measurements

The sorption/desorption profiles of the forms of the present invention were tested at 25° C. under 0-90% relative humidity. The forms of the present invention were classified according to the following criteria:

Deliquescent: Sufficient water is absorbed to form a liquid.

Very hygroscopic: Increase in mass is equal to or greater than 15%.

Hygroscopic: Increase in mass is less than 15% and equal to or greater than 2%.

Slightly hygroscopic: Increase in mass is less than 2% and equal to or greater than 0.2%.

Non-hygroscopic: Increase in mass is less than 0.2%.

5.2 Aqueous Solubility Measurements

Testing media: water, pH 1.2, 4.5, 6.8, 7.4 USP buffers, 0.01 N HCl, 0.1 N HCl, SGF, FaSSIF, FeSSIF.

The various testing media were prepared as follows:

pH 1.2 (USP): 50 mL of 0.2 M potassium chloride solution were placed in a 200 mL volumetric flask to which 85.0 mL of 0.2M hydrochloric acid solution were added followed by the addition of water to obtain the required volume.

pH 4.5 (USP): 50 mL of 0.2 M potassium biphthalate solution were placed in a 200 mL volumetric flask to which 8.8 mL of 0.2 M sodium hydroxide solution were added followed by the addition of water to obtain the required volume.

pH 6.8 (USP): 50 mL of 0.2 M monobasic potassium phosphate solution were placed in a 200 mL volumetric flask to which 22.4 mL of 0.2 M sodium hydroxide solution were added followed by the addition of water to obtain the required volume.

pH 7.4 (USP): 50 mL of 0.2 M monobasic potassium phosphate solution were placed in a 200 mL volumetric flask to which 39.1 mL of 0.2 M sodium hydroxide solution were added followed by the addition of water to obtain the required volume.

Simulated gastric fluid (SGF): 0.01 N HCl, 0.05% sodium lauryl sulfate, and 0.2% NaCl.

Fasted state simulated intestinal fluid (FaSSIF): 29 mM NaH₂PO₄, 3 mM Na taurocholate, 0.75 mM lecithin, 103 mM NaCl, and NaOH to obtain pH 6.5.

Fed state simulated intestinal fluid (FeSSIF): 144 mM acetic acid, 15 mM Na taurocholate, 3.75 mM lecithin, 204 mM NaCl, and NaOH to obtain pH 5.0.

Testing procedure: The tested febuxostat form was placed in each of the different media and was kept shaken for 24 hours at 25° C. Then, the saturated solution was filtered. The concentration of the febuxostat form in filtrate was determined by HPLC. The final pH was then tested. The test was conducted in duplication.

5.3 Solid Stability Measurements

Testing conditions: 40° C., 60° C., 40° C./RH 75%, 60° C./RH 75%, light

Testing procedure: About 3 mg of a febuxostat form were weighed in a glass vial and stored under the different conditions for 1 week and 2 weeks, separately. The same febuxostat form was stored at −20° C. as control. The test was conducted in duplication. The physical appearance, assay and total related substances of each of the febuxostat forms were measured by HPLC at the end of the first and second weeks.

5.4 Physical Stability Measurements

Testing conditions: 40° C., 60° C., 40° C./RH 75%, 60° C./RH 75%, light

Testing procedure: About 50 mg of a febuxostat form were weighed in glass vial for testing the physical stability and were stored under different conditions for 1 week and 2 weeks, separately. The same febuxostat form was stored at −20° C. as control. XRPD, DSC and TGA of each of the febuxostat forms were measured at the end of the first and second weeks.

5.5 Bulk and Tapped Density Measurements

Bulk density testing: A febuxostat form in an amount which is sufficient to complete the test was passed through a 1.0 mm (No. 18) screen to break up agglomerates that may have formed during storage. The febuxostat form was then weighed (M) and the powder was added into a 10 mL graduated cylinder. The powder was carefully leveled without compacting, and the unsettled apparent volume, Vo, was read. The bulk density, in g per mL, was calculated by the formula:

Bulk Density=M/Vo

Tapped density testing: A febuxostat form in an amount which is sufficient to complete the test was passed through a 1.0 mm (No. 18) screen to break up agglomerates that may have formed during storage. The febuxostat form was then weighed and the powder was added into a 10 mL graduated cylinder. The powder was carefully leveled without compacting. The cylinder was tapped 500 times initially and the tapped volume, Va, was measured to the nearest graduated unit. The tapping was then repeated for additional 750 times and the tapped volume, Vb, was measured to the nearest graduated unit. If the difference between the two volumes was less than 2%, Vb was taken as the final tapped volume, Vf. The tapped density, in g per mL, was calculated by the formula:

Tapped Density=M/Vf

### Example 2

### Hydrated Febuxostat Form II (Method 1)

General method 1 was performed. Thus, febuxostat API was dissolved in the following solvents/solvent mixtures: THF; THF:MeOH=1:1 (v/v); THF:EtOH=1:1 (v/v); THF:IPA=1:1 (v/v); THF:1-Butanol=1:1 (v/v) or THF:iPrOAc=1:1 (v/v). The solutions were then filtered through 0.22 μm filter into clean vessels. The solvents/solvent mixtures were evaporated at room temperatures to form febuxostat Form II. This polymorphic form was characterized by X-ray diffraction (FIG. 1, Table 1). FIG. 2 illustrates a characteristic DSC profile. FIG. 3 illustrates a characteristic TGA profile: 33-179° C.—weight loss of 3.86%; 186° C.-378° C.—weight loss of 95.36%. FIG. 4 illustrates a characteristic IR spectrum with peaks at about 658, 725, 766, 824, 912, 956, 1010, 1042, 1114, 1164, 1216, 1286, 1323, 1369, 1393, 1425, 1467, 1508, 1601, 1679, 1698, 2222, 2872, and 2958 cm⁻¹. FIG. 5 illustrates a characteristic FT-Raman spectrum with peaks at about 1028, 1050, 1175, 1303, 1328, 1375, 1431, 1513, 1578, 1607, 2232, and 2930 cm⁻¹.

TABLE 1

| Peak No. | 2-Theta | Intensity (%) |
|---|---|---|
| 1 | 4.799 | 60.3 |
| 2 | 6.860 | 5.0 |
| 3 | 8.339 | 1.5 |
| 4 | 9.602 | 8.8 |
| 5 | 11.740 | 100.0 |
| 6 | 13.722 | 1.6 |
| 7 | 15.641 | 31.1 |
| 8 | 16.680 | 4.2 |
| 9 | 17.620 | 1.6 |
| 10 | 19.899 | 3.6 |
| 11 | 23.659 | 6.3 |
| 12 | 25.161 | 2.4 |
| 13 | 28.722 | 1.8 |
| 14 | 29.960 | 1.3 |
| 15 | 34.260 | 2.3 |

### Example 3

### Febuxostat NMP Solvate Form IV (Method 2)

General method 2 was performed. Thus, febuxostat API was dissolved in the following solvents/mixture of solvents: NMP; 2-MeTHF:NMP=1:1 (v/v); DMF:NMP=1:1 (v/v); or NMP:THF=1:1 (v/v) at 50° C. The solutions were then cooled down at 5° C. to form crystals. Febuxostat NMP solvate (Form IV) has a molar ratio of 1:0.2 febuxostat:NMP. Febuxostat NMP solvate (Form IV) was characterized by X-ray diffraction (FIG. 6, Table 2). FIG. 7 illustrates a characteristic DSC profile. FIG. 8 illustrates a characteristic TGA profile: 33-163° C.—weight loss of 7.15%; 172° C.-371° C.—weight loss of 92.24%. FIG. 9 illustrates a characteristic IR spectrum with peaks at about 658, 725, 762, 826, 907, 952, 1010, 1037, 1129, 1164, 1217, 1283, 1319, 1370, 1397, 1426, 1467, 1509, 1604, 1682, 2227, 2872, and 2962 cm⁻¹. FIG. 10 illustrates a characteristic FT-Raman spectrum with peaks at about 155, 197, 326, 409, 467, 531, 836, 913, 1028, 1110, 1175, 1286, 1332, 1374, 1431, 1512, 1606, 1842, 1898, 2070, 2116, and 2232 cm⁻¹.

TABLE 2

| Peak No. | 2-Theta | Intensity (%) |
|---|---|---|
| 1 | 4.021 | 81.5 |
| 2 | 4.859 | 34.8 |
| 3 | 6.400 | 15.6 |
| 4 | 6.857 | 14.6 |
| 5 | 7.478 | 33.4 |
| 6 | 8.000 | 79.3 |
| 7 | 8.300 | 40.0 |
| 8 | 10.099 | 50.4 |
| 9 | 10.741 | 80.1 |
| 10 | 11.699 | 66.5 |
| 11 | 12.338 | 22.8 |
| 12 | 14.026 | 10.8 |
| 13 | 16.000 | 28.5 |
| 14 | 16.681 | 22.2 |
| 15 | 17.160 | 44.2 |
| 16 | 17.616 | 62.8 |
| 17 | 18.777 | 13.1 |
| 18 | 20.100 | 17.4 |
| 19 | 20.919 | 19.5 |
| 20 | 21.562 | 15.7 |
| 21 | 23.181 | 16.8 |
| 22 | 23.540 | 22.8 |
| 23 | 25.240 | 100.0 |
| 24 | 26.220 | 19.2 |

### Example 4

### Febuxostat NMP Solvate Form VI (Method 3)

General method 3 was performed. Thus, febuxostat API was dissolved in NMP to form a saturated solution at 25 or 50° C. An anti-solvent (either water or ACN) that was kept at 25° C. was then added to precipitate out crystals. The febuxostat NMP solvate prepared by this method has molar ratio of 1:0.5 febuxostat: NMP. Febuxostat NMP solvate (Form VI) was characterized by X-ray diffraction (FIG. 11, Table 3). FIG. 12 illustrates a characteristic DSC profile. FIG. 13 illustrates a characteristic TGA profile: 39-188° C.—weight loss of 12.43%; 193° C.-387° C.—weight loss of 87.08%. FIG. 14 illustrates a characteristic IR spectrum with peaks at about 657, 716, 745, 764, 824, 903, 948, 1007, 1042, 1091, 1128, 1170, 1223, 1262, 1295, 1372, 1393, 1428, 1471, 1508, 1604, 1682, 1699, 1728, 2222, 2868, and 2962 cm⁻¹. FIG. 15 illustrates a characteristic FT-Raman spectrum with peaks at about 1028, 1317, 1374, 1434, 1512, 1606, and 2229 cm⁻¹.

TABLE 3

| Peak No. | 2-Theta | Intensity (%) |
|---|---|---|
| 1 | 4.124 | 25.5 |
| 2 | 7.041 | 100.0 |
| 3 | 7.637 | 13.9 |
| 4 | 8.299 | 35.9 |
| 5 | 10.043 | 50.0 |
| 6 | 11.400 | 34.4 |
| 7 | 12.475 | 22.1 |
| 8 | 13.737 | 44.1 |
| 9 | 14.141 | 15.8 |
| 10 | 15.397 | 15.3 |
| 11 | 17.121 | 37.2 |
| 12 | 17.641 | 39.2 |
| 13 | 19.641 | 17.6 |
| 14 | 21.542 | 25.6 |
| 15 | 23.020 | 30.2 |
| 16 | 24.921 | 44.4 |
| 17 | 25.299 | 58.1 |
| 18 | 25.600 | 49.6 |
| 19 | 26.222 | 42.4 |
| 20 | 27.098 | 18.0 |
| 21 | 29.943 | 18.9 |

### Example 5

### Febuxostat DMSO Solvate Form V (Method 2)

General method 2 was performed. Thus, febuxostat API was dissolved in the following solvents/mixture of solvents: DMSO; 2-MeTHF:DMSO=1:1 (v/v); DMF:DMSO=1:1 (v/v); or NMP:DMSO=1:1 (v/v) at 50° C. The solutions were then cooled down at 5° C. to form crystals. Febuxostat DMSO solvate (Form V) has a molar ratio of 1:0.6 febuxostat:DMSO. Febuxostat DMSO solvate (Form V) was characterized by X-ray diffraction (FIG. 16, Table 4). FIG. 17 illustrates a characteristic DSC profile. FIG. 18 illustrates a characteristic TGA profile: 35-182° C.—weight loss of 15.17%; 188° C.-364° C.—weight loss of 84.34%. FIG. 19 illustrates a characteristic IR spectrum with peaks at about 653, 706, 743, 766, 827, 881, 907, 951, 1005, 1106, 1164, 1274, 1315, 1368, 1389, 1426, 1450, 1509, 1573, 1604, 1679, 2227, 2868, and 2966 cm⁻¹. FIG. 20 illustrates a characteristic FT-Raman spectrum with peaks at about 288, 337, 395, 433, 531, 578, 672, 708, 1041, 1323, 1371, 1452, 1512, 1574, 1609, and 1690 cm⁻¹.

TABLE 4

| Peak No. | 2-Theta | Intensity (%) |
|---|---|---|
| 1 | 7.119 | 100.0 |
| 2 | 10.616 | 8.3 |
| 3 | 11.679 | 4.5 |
| 4 | 13.840 | 15.0 |
| 5 | 14.318 | 27.9 |
| 6 | 15.240 | 5.1 |
| 7 | 16.158 | 69.1 |
| 8 | 16.864 | 4.6 |
| 9 | 17.204 | 6.2 |
| 10 | 19.438 | 7.4 |
| 11 | 21.039 | 6.4 |
| 12 | 21.580 | 7.2 |
| 13 | 21.763 | 5.2 |
| 14 | 22.142 | 5.8 |
| 15 | 22.498 | 8.7 |
| 16 | 22.721 | 6.4 |
| 17 | 23.539 | 46.6 |
| 18 | 24.779 | 9.1 |
| 19 | 26.400 | 41.3 |
| 20 | 28.719 | 5.3 |

When scaling up the preparation of the febuxostat DMSO solvate (Form V), about 1 g of febuxostat API was weighed into a vial. 1 mL of DMF:DMSO=1:1 was added followed by sonication for 5 minutes at 50° C. to form a clear solution. The solution was stored at room temperature for 30 minutes to form a precipitate. The residual solid was then separated by vacuum filtration and washed with ACN, followed by drying using a vacuum oven at 40° C. overnight. The febuxostat DMSO solvate (Form V) was characterized using X-ray powder diffraction (FIG. 39), DSC (FIG. 40) and TGA (FIG. 41).

### Example 6

### Febuxostat DMSO Solvate Form VII (Method 3)

General method 3 was performed. Thus, febuxostat API was dissolved in DMSO to form a saturated solution at 25 or 50° C. Water at 25° C. was then added as an anti-solvent to afford the precipitation of crystals. The febuxostat DMSO solvate prepared by this method has molar ratio of 1:0.8 febuxostat:DMSO. Febuxostat DMSO solvate (Form VII) was characterized by X-ray diffraction (FIG. 21, Table 5). FIG. 22 illustrates a characteristic DSC profile. FIG. 23 illustrates a characteristic TGA profile: 33-189° C.—weight loss of 17.03%; 189° C.-386° C.—weight loss of 82.77%. FIG. 24 illustrates a characteristic IR spectrum with peaks at about 653, 702, 743, 765, 827, 878, 951, 1009, 1106, 1160, 1274, 1315, 1368, 1389, 1422, 1450, 1509, 1605, 1680, 2222, 2872, and 2962 cm⁻¹. FIG. 25 illustrates a characteristic FT-Raman spectrum with peaks at about 357, 467, 531, 578, 675, 839, 1028, 1110, 1175, 1286, 1323, 1371, 1449, 1512, 1571, 1609, 1693, 1842, 2081, 2116, 2227, 2923, and 3502 cm⁻¹.

TABLE 5

| Peak No. | 2-Theta | Intensity (%) |
|---|---|---|
| 1 | 4.001 | 9.5 |
| 2 | 7.179 | 37.6 |
| 3 | 8.017 | 6.5 |
| 4 | 11.421 | 100.0 |
| 5 | 13.620 | 6.0 |
| 6 | 13.920 | 8.8 |
| 7 | 14.660 | 6.1 |
| 8 | 17.120 | 22.1 |
| 9 | 17.798 | 12.5 |
| 10 | 20.519 | 13.2 |
| 11 | 21.500 | 13.3 |
| 12 | 22.682 | 9.2 |
| 13 | 23.040 | 14.7 |
| 14 | 25.240 | 85.2 |
| 15 | 26.261 | 24.1 |
| 16 | 27.821 | 27.9 |

### Example 7

### Anhydrous Febuxostat Form VIII (Method 4)

General method 4 was performed. Thus, febuxostat API was heated to melt under vacuum. The melted compound was then rapidly or slowly cooled to afford the formation of febuxostat (Form VIII). Anhydrous febuxostat (Form VIII) was characterized by X-ray diffraction (FIG. 26, Table 6). FIG. 27 illustrates a characteristic DSC profile. FIG. 28 illustrates a characteristic TGA profile: 34-150° C.—weight loss of 27e-3%; 164° C.-374° C.—weight loss of 99.48%. FIG. 29 illustrates a characteristic IR spectrum with peaks at about 660, 725, 764, 824, 878, 910, 930, 1012, 1037, 1116, 1172, 1283, 1328, 1371, 1385, 1425, 1467, 1510, 1604, 1653, 1683, 2231, 2868, and 2958 cm⁻¹. FIG. 30 illustrates a characteristic FT-Raman spectrum with peaks at about 155, 239, 288, 347, 402, 467, 538, 605, 672, 748, 839, 913, 1009, 1100, 1175, 1286, 1326, 1374, 1434, 1512, 1609, 1664, 1768, 1864, 1898, 1973, 2070, 2235, 2272, and 2390 cm⁻¹.

TABLE 6

| Peak No. | 2-Theta | Intensity (%) |
|---|---|---|
| 1 | 3.642 | 84.5 |
| 2 | 7.083 | 18.9 |
| 3 | 12.381 | 100.0 |
| 4 | 13.261 | 39.9 |
| 5 | 17.581 | 9.1 |
| 6 | 23.120 | 17.2 |
| 7 | 25.180 | 82.0 |
| 8 | 26.959 | 56.1 |
| 9 | 27.579 | 30.9 |

### Example 8

### Anhydrous Febuxostat Form IX (Method 5)

General method 5 was performed. Thus, febuxostat API was dissolved in the following solvents: MeOH, MEK, acetone or EtOAc at room temperatures. The solvents were then removed by rotary evaporator below 50° C. Anhydrous febuxostat (Form IX) was characterized by X-ray diffraction (FIG. 31, Table 7). FIG. 32 illustrates a characteristic DSC profile. FIG. 33 illustrates a characteristic TGA profile: 33-76° C.—weight loss of 0.40%; 188° C.-326° C.—weight loss of 97.47%. FIG. 34 illustrates a characteristic IR spectrum with peaks at about 657, 715, 764, 825, 874, 911, 952, 1010, 1037, 1114, 1168, 1281, 1328, 1370, 1389, 1427, 1450, 1511, 1606, 1687, 2235, 2868 and 2962 cm⁻¹. FIG. 35 illustrates a characteristic FT-Raman spectrum with peaks at about 392, 467, 585, 748, 1047, 1175, 1332, 1374, 1431, 1512, 1609, 1842, 1892, 1973, 2081, and 2235 cm⁻¹.

TABLE 7

| Peak No. | 2-Theta | Intensity (%) |
|---|---|---|
| 1 | 4.599 | 4.4 |
| 2 | 6.099 | 26.3 |
| 3 | 7.337 | 5.5 |
| 4 | 9.199 | 4.6 |
| 5 | 11.620 | 61.4 |
| 6 | 13.300 | 3.0 |
| 7 | 16.259 | 5.7 |
| 8 | 17.280 | 44.3 |
| 9 | 18.480 | 4.3 |
| 10 | 23.000 | 5.5 |
| 11 | 25.721 | 100.0 |
| 12 | 26.460 | 25.6 |
| 13 | 28.280 | 3.8 |

When scaling up the preparation of anhydrous febuxostat (Form IX), about 1.3 g of febuxostat API were weighed into a round flask to which 20 ml of EtOAc were added followed by sonication for 5 minutes at room temperature to form a clear solution. The solvent was then removed by rotary evaporation below 50° C. The residual solid was dried using a vacuum oven at 40° C. overnight. The anhydrous febuxostat (Form IX) was characterized by X-ray diffraction (FIG. 42), DSC (FIG. 43) and TGA (FIG. 44).

### Example 9

### Febuxostat Hydrate Form XI (Method 6)

General method 6 was performed. Thus, about 1.2 g of febuxostat API were weighed into a vial. 20 ml of THF were then added. The vial was shaken by hand to form a clear solution. The THF was slowly evaporated in a fume hood at room temperature. The residual solid was further dried in a vacuum oven at 40° C. overnight.

Febuxostat hydrate (Form XI) was characterized by X-ray diffraction (FIG. 36, Table 8). FIG. 37 illustrates a characteristic DSC profile with an endothermic peak at about 199° C. FIG. 38 illustrates a characteristic TGA profile: 31-196° C.—weight loss of 1.54%. The results show that Form XI is a hydrate form with about 1.0% water content.

TABLE 8

| Peak No. | 2-Theta | Intensity (%) |
|---|---|---|
| 1 | 4.899 | 34.7 |
| 2 | 6.181 | 6.3 |
| 3 | 6.821 | 25.2 |
| 4 | 8.178 | 4.9 |
| 5 | 9.741 | 5.7 |
| 6 | 11.582 | 100.0 |
| 7 | 12.162 | 14.3 |
| 8 | 13.621 | 6.7 |
| 9 | 15.802 | 23.9 |
| 10 | 16.299 | 12.5 |
| 11 | 17.458 | 20.3 |
| 12 | 19.436 | 4.2 |
| 13 | 20.459 | 5.0 |
| 14 | 21.281 | 5.4 |
| 15 | 21.540 | 6.1 |
| 16 | 23.177 | 7.5 |
| 17 | 24.841 | 29.7 |
| 18 | 25.220 | 56.8 |
| 19 | 25.796 | 30.0 |
| 20 | 26.160 | 24.1 |
| 21 | 26.777 | 12.6 |
| 22 | 27.781 | 6.4 |
| 23 | 29.220 | 6.2 |
| 24 | 29.841 | 4.4 |

### Example 10

### Physical and Chemical Properties of Febuxostat Forms V, IX and XI

Febuxostat forms V, IX and XI prepared according to Examples 5 (scale-up), 8 (scale-up) and 9, respectively, were characterized for assessing their physical and chemical properties and were further compared with Form G of U.S. Pat. No. 6,225,474.

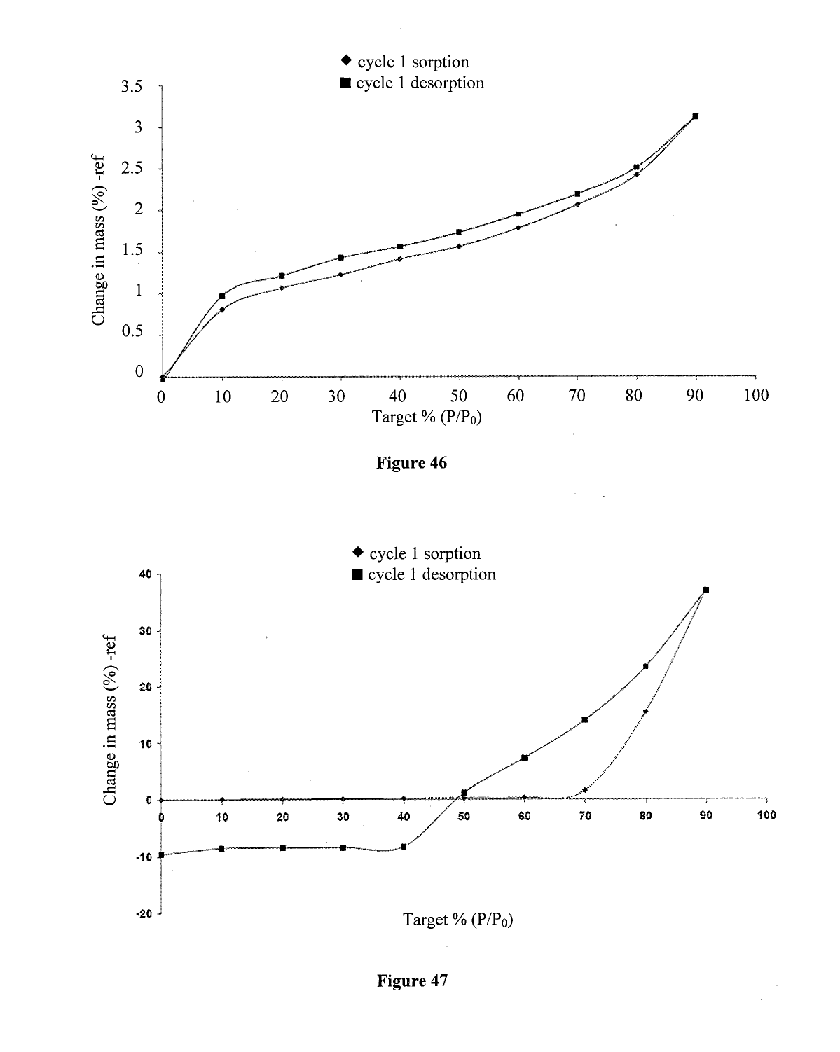

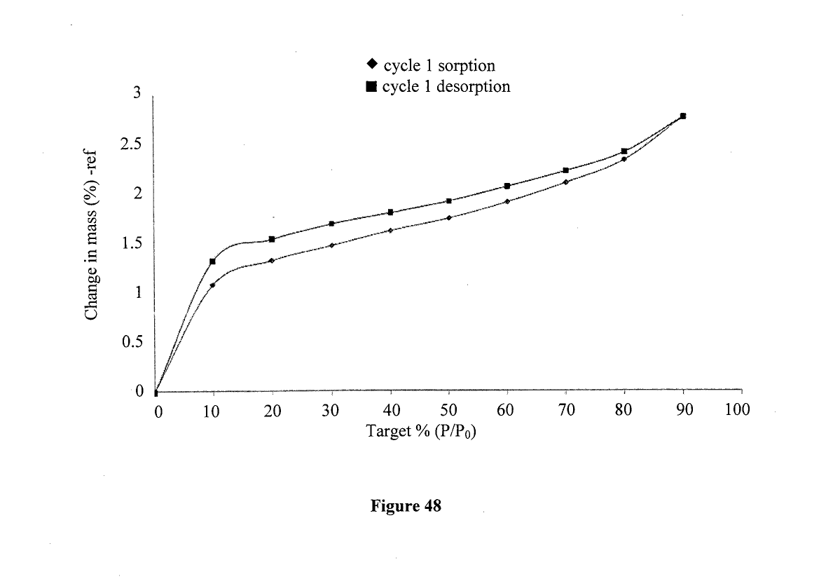

The DVS isotherm plots of the febuxostat forms are shown in FIGS. 45-48 and are summarized in Table 9. The different forms are classified as follows: anhydrous febuxostat (Form IX) is classified as hygroscopic (FIG. 45) and febuxostat hydrate (Form XI) is classified as hygroscopic (FIG. 46). The febuxostat DMSO solvate (Form V) is classified as very hygroscopic with about 10% weight loss after sorption-desorption cycle which might be attributed to DMSO evaporation at high humidity conditions. Form IX is less hygroscopic than Form G of U.S. Pat. No. 6,225,474 (FIG. 48).

TABLE 9

| | Target | Change in Mass (%) - ref | | |
|---|---|---|---|---|
| Form | % P/P₀ | Sorption | Desorption | Hysteresis |
| Form G of U.S. | 0.0 | 0.000 | −0.013 | |
| Pat. No. 6,225,474 | 10.0 | 1.082 | 1.323 | 0.241 |
| | 20.0 | 1.326 | 1.540 | 0.215 |
| | 30.0 | 1.473 | 1.692 | 0.219 |
| | 40.0 | 1.625 | 1.806 | 0.181 |
| | 50.0 | 1.750 | 1.923 | 0.173 |
| | 60.0 | 1.915 | 2.074 | 0.158 |
| | 70.0 | 2.109 | 2.229 | 0.119 |
| | 80.0 | 2.342 | 2.420 | 0.078 |
| | 90.0 | 2.776 | 2.776 | |
| Form V | 0.0 | 0.00 | −9.66 | |
| | 10.0 | 0.08 | −8.56 | −8.64 |
| | 20.0 | 0.12 | −8.49 | −8.60 |
| | 30.0 | 0.14 | −8.40 | −8.53 |
| | 40.0 | 0.20 | −8.30 | −8.50 |
| | 50.0 | 0.23 | 1.16 | 0.93 |
| | 60.0 | 0.31 | 7.34 | 7.02 |
| | 70.0 | 1.59 | 14.03 | 12.45 |
| | 80.0 | 15.44 | 23.42 | 7.98 |
| | 90.0 | 36.92 | 36.92 | |
| Form IX | 0.0 | 0.000 | 0.031 | |
| | 10.0 | 0.127 | 0.247 | 0.121 |
| | 20.0 | 0.195 | 0.323 | 0.128 |
| | 30.0 | 0.252 | 0.428 | 0.176 |
| | 40.0 | 0.351 | 0.510 | 0.160 |
| | 50.0 | 0.432 | 0.616 | 0.184 |
| | 60.0 | 0.566 | 0.786 | 0.221 |
| | 70.0 | 0.747 | 1.139 | 0.391 |
| | 80.0 | 1.000 | 1.583 | 0.583 |
| | 90.0 | 2.135 | 2.135 | |
| Form XI | 0.0 | 0.001 | −0.052 | |
| | 10.0 | 0.810 | 0.973 | 0.164 |
| | 20.0 | 1.064 | 1.210 | 0.146 |
| | 30.0 | 1.222 | 1.425 | 0.203 |
| | 40.0 | 1.410 | 1.560 | 0.150 |
| | 50.0 | 1.559 | 1.728 | 0.169 |
| | 60.0 | 1.782 | 1.948 | 0.166 |
| | 70.0 | 2.057 | 2.187 | 0.130 |
| | 80.0 | 2.414 | 2.503 | 0.089 |
| | 90.0 | 3.111 | 3.111 | |

Febuxostat Forms V, IX and XI of the present invention show good solubility in pH6.8 USP buffer, pH7.4 USP buffer, FaSSIF, FeSSIF and show poor solubility in pH1.2 USP buffer, 0.01N HCl, 0.1N HCl and SGF (Table 10; aqueous solubility). Without being bound by any theory or mechanism of action, the improved solubility of the febuxostat forms of the present invention in basic media suggests better febuxostat absorption is the colon where the pH ranges form 6.8 to 7.4. As the major site of febuxostat absorption is the colon, the solubility measurements imply improved bioavailability of the febuxostat polymorphs of the present invention.

TABLE 10

| | | | | HPLC |
|---|---|---|---|---|
| | | | | solubility |
| Form | Media | pH | Visual solubility | (μg/mL) |
| Form G | water | 8.416 | Turbid + few particles | 29.80 |
| of U.S. | pH 1.2 (USP) | 1.089 | few particles | <LOD¹ |
| Pat. No. | pH 4.5 (USP) | 4.469 | few particles | 0.11 |
| 6,225,474 | pH 6.8 (USP) | 6.686 | Turbid + few particles | 764.90 |
| | pH 7.4 (USP) | 6.985 | Slightly Turbid + few | 3325.37 |
| | | | particles | |
| | 0.01N HCl | 1.987 | few particles | <LOD¹ |
| | 0.1N HCl | 0.945 | few particles | <LOD¹ |
| | SGF | 2.118 | few particles | <LOD¹ |
| | FaSSIF | 6.384 | Turbid + few particles | 448.35 |
| | FeSSIF | 4.965 | Turbid + few particles | 132.21 |
| Form V | water | 7.520 | Turbid + few particles | 54.85 |
| | pH 1.2 (USP) | 1.157 | few particles | <LOD³ |
| | pH 4.5 (USP) | 4.452 | Turbid + few particles | 0.38 |
| | pH 6.8 (USP) | 6.641 | Turbid + few particles | 1123.78 |
| | pH 7.4 (USP) | 6.960 | Slightly turbid | 4317.12 |
| | 0.01N HCl | 2.003 | few particles | <LOD³ |
| | 0.1N HCl | 0.951 | few particles | <LOD³ |
| | SGF | 2.108 | few particles | <LOD³ |
| | FaSSIF | 6.384 | Turbid + few particles | 513.37 |
| | FeSSIF | 4.995 | Turbid + few particles | 180.61 |
| Form IX | water | 5.403 | Turbid + few particles | 32.99 |
| | pH 1.2 (USP) | 1.242 | Turbid + few particles | <LOD⁴ |
| | pH 4.5 (USP) | 4.524 | Turbid + few particles | 0.78 |
| | pH 6.8 (USP) | 6.477 | Turbid + few particles | 1409.99 |
| | pH 7.4 (USP) | 6.690 | Slightly turbid | 4836.10 |
| | 0.01N HCl | 2.078 | Turbid + few particles | <LOD⁴ |
| | 0.1N HCl | 1.060 | Turbid + few particles | <LOD⁴ |
| | SGF | 2.097 | Turbid | <LOD⁴ |
| | FaSSIF | 6.246 | Turbid | 606.65 |
| | FeSSIF | 4.983 | Turbid | 192.53 |
| Form XI | water | 8.106 | Turbid + few particles | 33.82 |
| | pH 1.2 (USP) | 1.132 | few particles | <LOD² |
| | pH 4.5 (USP) | 4.479 | few particles | 0.49 |
| | pH 6.8 (USP) | 6.684 | Turbid + few particles | 931.90 |
| | pH 7.4 (USP) | 6.905 | Slightly turbid + few | 4464.94 |
| | | | particles | |
| | 0.01N HCl | 1.981 | few particles | <LOD² |
| | 0.1N HCl | 0.967 | few particles | <LOD² |
| | SGF | 2.114 | few particles | <LOD² |
| | FaSSIF | 6.336 | Turbid + few particles | 521.19 |
| | FeSSIF | 4.998 | Turbid + few particles | 149.95 |

LOD¹ = 43.20 ng/ml; LOD² = 43.17 ng/ml; LOD³ = 77.63 ng/ml; LOD⁴ = 26.40 ng/ml

The solid stability of the febuxostat forms of the present invention under various conditions was measured and the results are summarized in Tables 11-13. The assay and TRS of febuxostat DMSO solvate (Form V), anhydrous febuxostat (Form IX), and febuxostat hydrate (Form XI) show no significant change under different conditions (40° C., 60° C., 40° C./75% RH, 60° C./75% RH) at end of the first and second weeks. When stored under light the recovery of all of the forms at end of the first and second week was 92.8%-97.5%, and the TRS increased by 1.7%-6.5%.

TABLE 11
Febuxostat DMSO solvate (Form V)

| | | | | | | | | Cal. | |
|---|---|---|---|---|---|---|---|---|---|
| | | | Weight | TRS | | Volume | Peak | Weight | Remaining |
| Condition | Time | Sample | (mg) | (%) | Appearance | (mL) | Area | (mg) | (%) |
| −20° C. | 7 d | 1 | 3.000 | 0.40 | No change | 10 | 8385.9 | — | — |
| | | 2 | 3.194 | 0.39 | No change | 10 | 8702.9 | — | |
| | 14 d | 1 | 3.227 | 0.41 | No change | 10 | 8752.2 | — | — |
| | | 2 | 2.994 | 0.42 | No change | 10 | 8161.5 | — | |
| 40° C. | 7 d | 1 | 3.002 | 0.45 | No change | 10 | 8206.6 | 3.012 | 100.62 |
| | | 2 | 2.938 | 0.45 | No change | 10 | 8288.1 | 2.965 | |
| | 14 d | 1 | 3.085 | 0.46 | No change | 10 | 8346.3 | 3.077 | 100.01 |
| | | 2 | 3.087 | 0.47 | No change | 10 | 8394.7 | 3.095 | |
| 60° C. | 7 d | 1 | 3.067 | 0.53 | No change | 10 | 8435.4 | 3.096 | 100.65 |
| | | 2 | 3.015 | 0.51 | No change | 10 | 8244.5 | 3.026 | |
| | 14 d | 1 | 3.077 | 0.54 | No change | 10 | 8305.6 | 3.062 | 100.06 |
| | | 2 | 2.955 | 0.53 | No change | 10 | 8063.1 | 2.973 | |
| 40° C./75% | 7 d | 1 | 3.180 | 0.47 | No change | 10 | 8553.9 | 3.139 | 99.40 |
| RH | | 2 | 3.102 | 0.48 | No change | 10 | 8458.6 | 3.104 | |
| | 14 d | 1 | 3.248 | 0.50 | No change | 10 | 8640.9 | 3.186 | 99.03 |
| | | 2 | 3.073 | 0.52 | No change | 10 | 8332.2 | 3.072 | |
| 60° C./75% | 7 d | 1 | 3.200 | 0.51 | No change | 10 | 8549.9 | 3.138 | 98.20 |
| RH | | 2 | 3.188 | 0.52 | No change | 10 | 8542.5 | 3.135 | |
| | 14 d | 1 | 3.017 | 0.52 | No change | 10 | 8133.8 | 2.999 | 98.87 |
| | | 2 | 3.158 | 0.53 | No change | 10 | 8422.5 | 3.105 | |
| Light | 7 d | 1 | 2.963 | 1.02 | Light yellow | 10 | 8107.0 | 2.900 | 97.74 |
| | | 2 | 3.021 | 1.18 | Light yellow | 10 | 8241.9 | 2.948 | |
| | 14 d | 1 | 3.220 | 5.99 | Light yellow | 10 | 8145.2 | 3.003 | 94.06 |
| | | 2 | 3.072 | 5.08 | Light yellow | 10 | 7903.3 | 2.914 | |

TABLE 12
Anhydrous Febuxostat (Form IX)

| | | | | | | | | Cal. | |
|---|---|---|---|---|---|---|---|---|---|
| | | | Weight | TRS | | Volume | Peak | Weight | Remaining |
| Condition | Time | Sample | (mg) | (%) | Appearance | (mL) | Area | (mg) | (%) |
| −20° C. | 7 d | 1 | 3.198 | 1.23 | No change | 10 | 9879.4 | — | — |
| | | 2 | 3.087 | 1.22 | No change | 10 | 9570.6 | — | |
| | 14 d | 1 | 3.193 | 1.23 | No change | 10 | 9931.2 | — | — |
| | | 2 | 3.032 | 1.24 | No change | 10 | 9674.4 | — | |
| 40° C. | 7 d | 1 | 3.271 | 1.28 | No change | 10 | 10092.7 | 3.267 | 99.70 |
| | | 2 | 3.322 | 1.30 | No change | 10 | 10212.9 | 3.306 | |
| | 14 d | 1 | 3.159 | 1.29 | No change | 10 | 9922.1 | 3.190 | 100.02 |
| | | 2 | 3.126 | 1.32 | No change | 10 | 9631.8 | 3.097 | |
| 60° C. | 7 d | 1 | 2.996 | 1.37 | No change | 10 | 9265.4 | 2.999 | 100.80 |
| | | 2 | 3.082 | 1.35 | No change | 10 | 9662.8 | 3.128 | |
| | 14 d | 1 | 3.138 | 1.38 | No change | 10 | 9789.9 | 3.148 | 100.41 |
| | | 2 | 3.071 | 1.35 | No change | 10 | 9600.8 | 3.087 | |
| 40° C./75% | 7 d | 1 | 3.216 | 1.34 | No change | 10 | 10103.2 | 3.270 | 100.47 |
| RH | | 2 | 3.283 | 1.31 | No change | 10 | 10064.9 | 3.258 | |
| | 14 d | 1 | 3.283 | 1.35 | No change | 10 | 10162.2 | 3.267 | 99.47 |
| | | 2 | 3.124 | 1.33 | No change | 10 | 9659.5 | 3.106 | |
| 60° C./75% | 7 d | 1 | 3.313 | 1.33 | No change | 10 | 10307.3 | 3.337 | 100.89 |
| RH | | 2 | 3.212 | 1.33 | No change | 10 | 10028.2 | 3.246 | |
| | 14 d | 1 | 3.194 | 1.35 | No change | 10 | 9812.2 | 3.155 | 100.07 |
| | | 2 | 3.077 | 1.33 | No change | 10 | 9952.1 | 3.119 | |
| Light | 7 d | 1 | 3.208 | 3.07 | Light yellow | 10 | 9669.3 | 3.130 | 97.50 |
| | | 2 | 3.356 | 3.05 | Light yellow | 10 | 10100.6 | 3.270 | |
| | 14 d | 1 | 3.218 | 6.30 | Light yellow | 10 | 9292.8 | 2.988 | 92.81 |
| | | 2 | 3.228 | 6.54 | Light yellow | 10 | 9315.2 | 2.995 | |

TABLE 13
Crystalline Febuxostat Hydrate (Form XI)

| | | | | | | | | Cal. | |
|---|---|---|---|---|---|---|---|---|---|
| | | | Weight | TRS | | Volume | Peak | Weight | Remaining |
| Condition | Time | Sample | (mg) | (%) | Appearance | (mL) | Area | (mg) | (%) |
| −20° C. | 7 d | 1 | 3.075 | 1.22 | No change | 10 | 9510.1 | — | — |
| | | 2 | 3.013 | 1.22 | No change | 10 | 9466.7 | — | |
| | 14 d | 1 | 3.220 | 1.23 | No change | 10 | 9665.3 | — | — |
| | | 2 | 3.301 | 1.23 | No change | 10 | 9834.4 | — | |
| 40° C. | 7 d | 1 | 3.043 | 1.25 | No change | 10 | 9358.9 | 3.026 | 99.02 |
| | | 2 | 3.086 | 1.24 | No change | 10 | 9409.9 | 3.043 | |
| | 14 d | 1 | 3.180 | 1.26 | No change | 10 | 9587.2 | 3.194 | 100.12 |
| | | 2 | 3.307 | 1.27 | No change | 10 | 9907.6 | 3.301 | |
| 60° C. | 7 d | 1 | 3.127 | 1.31 | No change | 10 | 9513.9 | 3.076 | 98.56 |
| | | 2 | 3.178 | 1.30 | No change | 10 | 9704.8 | 3.138 | |
| | 14 d | 1 | 3.275 | 1.33 | No change | 10 | 9823.4 | 3.273 | 99.72 |
| | | 2 | 3.302 | 1.33 | No change | 10 | 9863.8 | 3.286 | |
| 40° C./75% | 7 d | 1 | 3.142 | 1.28 | No change | 10 | 9688.3 | 3.133 | 99.02 |
| RH | | 2 | 3.125 | 1.28 | No change | 10 | 9504.9 | 3.073 | |
| | 14 d | 1 | 3.272 | 1.30 | No change | 10 | 9851.3 | 3.282 | 100.75 |
| | | 2 | 3.178 | 1.29 | No change | 10 | 9654.0 | 3.216 | |
| 60° C./75% | 7 d | 1 | 3.272 | 1.33 | No change | 10 | 9972.7 | 3.225 | 98.44 |
| RH | | 2 | 3.197 | 1.31 | No change | 10 | 9722.5 | 3.144 | |
| | 14 d | 1 | 3.301 | 1.35 | No change | 10 | 9779.8 | 3.258 | 99.82 |
| | | 2 | 3.222 | 1.36 | No change | 10 | 9761.9 | 3.252 | |
| Light | 7 d | 1 | 3.103 | 1.86 | Light yellow | 10 | 9242.3 | 2.988 | 96.26 |
| | | 2 | 3.047 | 2.09 | Light yellow | 10 | 9067.3 | 2.932 | |
| | 14 d | 1 | 3.230 | 4.88 | Light yellow | 10 | 9217.0 | 3.071 | 95.06 |
| | | 2 | 3.295 | 4.32 | Light yellow | 10 | 9401.1 | 3.132 | |

The physical stability of the febuxostat forms of the present invention under various conditions (40° C., 60° C., 40° C./75% RH, 60° C./75% RH and light) at end of the first and second weeks was further measured using XRPD, DSC and TGA. Febuxostat forms V, IX and XI of the present invention were stable under 40° C., 60° C. and light at the end of the first and second week. Febuxostat DMSO solvate (Form V) partially converted to form G of U.S. Pat. No. 6,225,474 under 40° C./75% RH at the end of the first week and completely converted to form G of U.S. Pat. No. 6,225,474 under 40° C./75% RH at the end of the second week. Anhydrous febuxostat (Form IX) partially converted to form G of U.S. Pat. No. 6,225,474 under 40° C./75% RH at the end of second week. Forms V, IX and XI completely converted to form G of U.S. Pat. No. 6,225,474 under 60° C./75% RH at the end of the first and second weeks. Febuxostat hydrate (Form XI) completely converted to form G of U.S. Pat. No. 6,225,474 under 40° C./75% RH at the end of the first and second weeks.

The bulk and tapped density of febuxostat forms V, IX and XI of the present invention were measured and compared to form G of U.S. Pat. No. 6,225,474. The results are summarized in Table 14.

TABLE 14

| Form | Bulk Density (g/ml) | Tapped Density (g/ml) |
|---|---|---|
| Form G of U.S. | 0.376 | 0.511 |
| Pat. No. 6,225,474 | | |
| Form V | 0.444 | 0.637 |
| Form IX | 0.177 | 0.282 |
| Form XI | 0.346 | 0.553 |

Anhydrous febuxostat (Form IX) of the present invention has the lowest bulk and tapped densities and can thus be easily formulated as tablets. Febuxostat forms V and XI have adequate bulk and tapped densities which allow for easy incorporation into a variety of different formulations.

### Example 11

### Preparation and Characterization of Febuxostat DMSO Solvate (Form V) Single Crystal

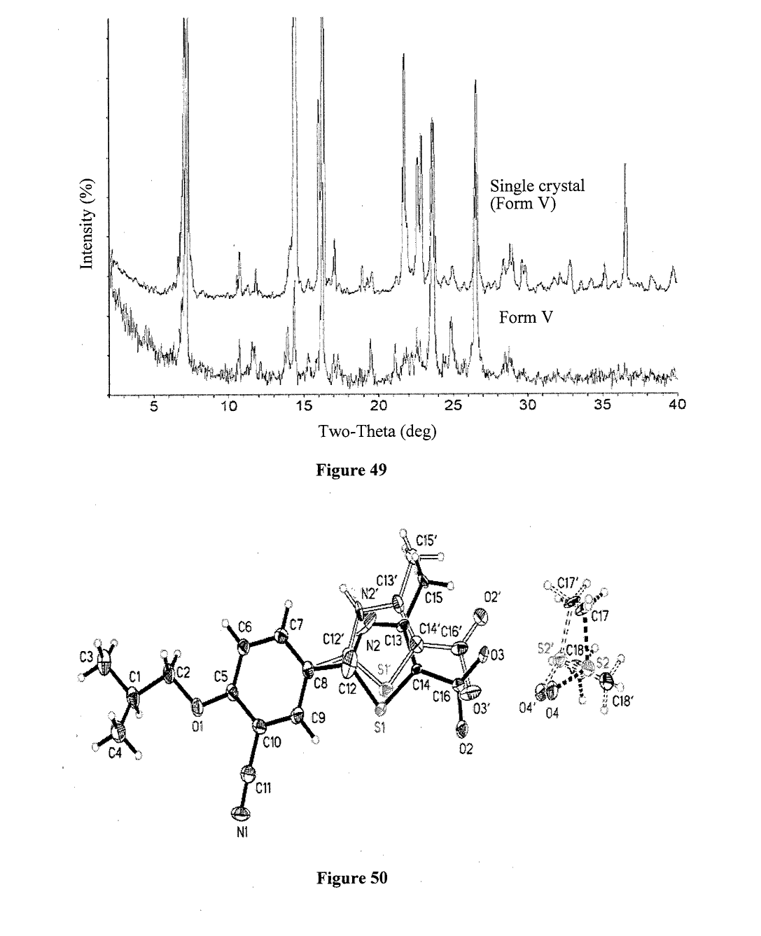

About 200 mg of febuxostat API were weighed into a vial. 400 μL of DMF:DMSO=1:1 were added into the vial followed by 5 minutes sonication at 50° C. to obtain a clear solution. The solution was stored at room temperature for 30 minutes with no crystal precipitation. About 1 mg of febuxostat DMSO solvate (Form V) was added and precipitation occurred. The mixture was sonicated for 5 minutes at 50° C. to obtain a clear solution. The solution was stored at room temperature for 4-5 days. A single crystal of febuxostat DMSO solvate (Form V) was formed. The single crystal was first analyzed by XRPD (FIG. 49). The single crystal was then analyzed using Bruker Smart DUO X-Ray single crystal diffraction (voltage: 50 kV, current: 30 mA, temperature: −140° C.). The space group was determined as P-1 with the following cell dimensions: a=7.659, b=10.608, c=12.746, a=85.393, P=74.852, y=83.845. The results are shown in FIG. 50.

### Example 12

### Formulation of Febuxostat Form IX

About 100 g of anhydrous febuxostat (Form IX) are mixed with about 300 g of lactose, 100 g of starch and 10 g of hydroxypropyl cellulose. The mixture is then charged into a mixer granulator with addition of DDW quantum satis to obtain granules which are consequently dried in a fluid bed drier at 60° C. The produced granules are sieved to remove particles having a size larger than 700 microns. The sieved granules are mixed with 25 g of crosscarmellose sodium and 5 g of magnesium stearate in a cross rotary mixer to obtain the lubricated granules. The lubricated granules are tableted with a rotary type tableting machine using a tableting pressure of 2,500 kgf/cm³.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-53. (canceled)

54. A crystalline anhydrous febuxostat (Form IX) having an X-ray powder diffraction pattern with diffraction peaks at 2-theta values of about 4.6±0.1, 6.1±0.1, 7.3±0.1, 9.2±0.1, 11.6±0.1, 13.3±0.1, 16.3±0.1, 17.3±0.1, 18.5±0.1, 23.0±0.1, 25.7±0.1, 26.5±0.1 and 28.3±0.1.

55. The crystalline anhydrous febuxostat (Form IX) according to claim 54 having an X-ray powder diffraction pattern substantially as shown in any of FIG. 31 or 42.

56. The crystalline anhydrous febuxostat (Form IX) according to claim 54, further characterized by:
(a) a DSC profile substantially as shown in any of FIG. 32 or 43; or
(b) TGA profile substantially as shown in any of FIG. 33 or 44; or
(c) an IR spectrum substantially as shown in FIG. 34; or
(d) an FT-Raman spectrum substantially as shown in FIG. 35.

57. The crystalline anhydrous febuxostat (Form IX) according to claim 56,
wherein the IR spectrum has characteristic peaks at about 657±4, 715±4, 764±4, 825±4, 874±4, 911±4, 952±4, 1010±4, 1037±4, 1114±4, 1168±4, 1281±4, 1328±4, 1370±4, 1389±4, 1427±4, 1450±4, 1511±4, 1606±4, 1687±4, 2235±4, 2868±4 and 2962±4 cm⁻¹; or
wherein the FT-Raman spectrum has characteristic peaks at about 392±4, 467±4, 585±4, 748±4, 1047±4, 1175±4, 1332±4, 1374±4, 1431±4, 1512±4, 1609±4, 1842±4, 1892±4, 1973±4, 2081±4, and 2235±4 cm⁻¹.

58. A process for preparing the crystalline anhydrous febuxostat (Form IX) according to claim 54, comprising the steps of:
(a) dissolving febuxostat in a solvent selected from MeOH, MEK, acetone, and EtOAc; and
(b) rapidly evaporating the solvent so as to precipitate crystalline anhydrous febuxostat (Form IX),
the process optionally further comprising the step of drying the febuxostat (Form IX) obtained in step (b) under vacuum.

59. A crystalline febuxostat NMP solvate (Form IV) having an X-ray powder diffraction pattern with diffraction peaks at 2-theta values of about 4.0±0.1, 4.9±0.1, 6.4±0.1, 6.9±0.1, 7.5±0.1, 8.0±0.1, 8.3±0.1, 10.1±0.1, 10.7±0.1, 11.7±0.1, 12.3±0.1, 14.0±0.1, 16.0±0.1, 16.7±0.1, 17.2±0.1, 17.6±0.1, 18.8±0.1, 20.1±0.1, 20.9±0.1, 21.6±0.1, 23.2±0.1, 23.6±0.1, 25.2±0.1, and 26.2±0.1.

60. The crystalline febuxostat NMP solvate (Form IV) according to claim 59, having an X-ray powder diffraction pattern substantially as shown in FIG. 6.

61. The crystalline febuxostat NMP solvate (Form IV) according to claim 59, further characterized by
(a) a DSC profile substantially as shown in FIG. 7; or
(b) a TGA profile substantially as shown in FIG. 8; or
(c) an IR spectrum substantially as shown in FIG. 9; or
(d) an FT-Raman spectrum substantially as shown in FIG. 10.

62. The crystalline febuxostat NMP solvate (Form IV) according to claim 61,
wherein the IR spectrum has characteristic peaks at about 658±4, 725±4, 762±4, 826±4, 907±4, 952±4, 1010±4, 1037±4, 1129±4, 1164±4, 1217±4, 1283±4, 1319±4, 1370±4, 1397±4, 1426±4, 1467±4, 1509±4, 1604±4, 1682±4, 2227±4, 2872±4, and 2962±4 cm⁻¹; or
wherein the FT-Raman spectrum has characteristic peaks at about 155±4, 197±4, 326±4, 409±4, 467±4, 531±4, 836±4, 913±4, 1028±4, 1110±4, 1175±4, 1286±4, 1332±4, 1374±4, 1431±4, 1512±4, 1606±4, 1842±4, 1898±4, 2070±4, 2116±4, and 2232±4 cm⁻¹.

63. A process for preparing the crystalline febuxostat NMP solvate (Form IV) according to claim 59, comprising the steps of:
(a) dissolving febuxostat in a solvent or a mixture of solvents selected from NMP, 2-MeTHF:NMP, DMF:NMP, and NMP:THF, optionally under heat; and
(b) cooling the solution obtained in step (a) so as to precipitate crystalline febuxostat NMP solvate (Form IV).

64. A crystalline febuxostat NMP solvate (Form VI) having an X-ray powder diffraction pattern with diffraction peaks at 2-theta values of about 4.1±0.1, 7.0±0.1, 7.6±0.1, 8.3±0.1, 10.0±0.1, 11.4±0.1, 12.5±0.1, 13.7±0.1, 14.1±0.1, 15.4±0.1, 17.1±0.1, 17.6±0.1, 19.6±0.1, 21.5±0.1, 23.0±0.1, 24.9±0.1, 25.3±0.1, 25.6±0.1, 26.2±0.1, 27.1±0.1, and 29.9±0.1.

65. The crystalline febuxostat NMP solvate (Form VI) according to claim 64, having an X-ray powder diffraction pattern substantially as shown in FIG. 11.

66. The crystalline febuxostat NMP solvate (Form VI) according to claim 64, further characterized by
(a) a DSC profile substantially as shown in FIG. 12; or
(b) a TGA profile substantially as shown in FIG. 13; or
(c) an IR spectrum substantially as shown in FIG. 14; or
(d) an FT-Raman spectrum substantially as shown in FIG. 15.

67. The crystalline febuxostat NMP solvate (Form VI) according to claim 66,
wherein the IR spectrum has characteristic peaks at about 657±4, 716±4, 745±4, 764±4, 824±4, 903±4, 948±4, 1007±4, 1042±4, 1091±4, 1128±4, 1170±4, 1223±4, 1262±4, 1295±4, 1372±4, 1393±4, 1428±4, 1471±4, 1508±4, 1604±4, 1682±4, 1699±4, 1728±4, 2222±4, 2868±4, and 2962±4 cm⁻¹; or
wherein the FT-Raman spectrum has characteristic peaks at about 1028±4, 1317±4, 1374±4, 1434±4, 1512±4, 1606±4, and 2229±4 cm⁻¹.

68. A process for preparing the crystalline febuxostat NMP solvate (Form VI) according to claim 64, comprising the steps of:
(a) dissolving febuxostat in NMP, optionally under heat; and
(b) adding an anti-solvent selected from water and ACN so as to precipitate crystalline febuxostat NMP solvate (Form VI).

69. A crystalline febuxostat DMSO solvate (Form VII) having an X-ray powder diffraction pattern with diffraction peaks at 2-theta values of about 4.0±0.1, 7.2±0.1, 8.0±0.1, 11.4±0.1, 13.6±0.1, 13.9±0.1, 14.7±0.1, 17.1±0.1, 17.8±0.1, 20.5±0.1, 21.5±0.1, 22.7±0.1, 23.0±0.1, 25.2±0.1, 26.3±0.1, and 27.8±0.1.

70. The crystalline febuxostat DMSO solvate (Form VII) according to claim 69, having an X-ray powder diffraction pattern substantially as shown in FIG. 21.

71. The crystalline febuxostat DMSO solvate (Form VII) according to claim 69, further characterized by
(a) a DSC profile substantially as shown in FIG. 22; or
(b) a TGA profile substantially as shown in FIG. 23; or
(c) an IR spectrum substantially as shown in FIG. 24; or
(d) an FT-Raman spectrum substantially as shown in FIG. 25.

72. The crystalline febuxostat DMSO solvate (Form VII) according to claim 71,
wherein the IR spectrum has characteristic peaks at about 653±4, 702±4, 743±4, 765±4, 827±4, 878±4, 951±4, 1009±4, 1106±4, 1160±4, 1274±4, 1315±4, 1368±4, 1389±4, 1422±4, 1450±4, 1509±4, 1605±4, 1680±4, 2222±4, 2872±4, and 2962±4 cm⁻¹; or
wherein the FT-Raman spectrum has characteristic peaks at about 357±4, 467±4, 531±4, 578±4, 675±4, 839±4, 1028±4, 1110±4, 1175±4, 1286±4, 1323±4, 1371±4, 1449±4, 1512±4, 1571±4, 1609±4, 1693±4, 1842±4, 2081±4, 2116±4, 2227±4, 2923±4, and 3502±4 cm⁻¹.

73. A process for preparing the crystalline febuxostat DMSO solvate (Form VII) according to claim 69, comprising the steps of:
(a) dissolving febuxostat in DMSO, optionally under heat; and
(b) adding an anti-solvent, wherein the anti-solvent is ACN so as to precipitate crystalline febuxostat DMSO solvate (Form VII).

74. A crystalline anhydrous febuxostat (Form VIII) having an X-ray powder diffraction pattern with diffraction peaks at 2-theta values of about 3.6±0.1, 7.1±0.1, 12.4±0.1, 13.3±0.1, 17.6±0.1, 23.1±0.1, 25.2±0.1, 27.0±0.1, and 27.6±0.1.

75. The crystalline anhydrous febuxostat (Form VIII) according to claim 74, having an X-ray powder diffraction pattern substantially as shown in FIG. 26.

76. The crystalline anhydrous febuxostat (Form VIII) according to claim 74, further characterized by
(a) a DSC profile substantially as shown in FIG. 27; or
(b) a TGA profile substantially as shown in FIG. 28; or
(c) an IR spectrum substantially as shown in FIG. 29; or
(d) an FT-Raman spectrum substantially as shown in FIG. 30.

77. The crystalline anhydrous febuxostat (Form VIII) according to claim 76,
wherein the IR spectrum has characteristic peaks at about 660±4, 725±4, 764±4, 824±4, 878±4, 910±4, 930±4, 1012±4, 1037±4, 1116±4, 1172±4, 1283±4, 1328±4, 1371±4, 1385±4, 1425±4, 1467±4, 1510±4, 1604±4, 1653±4, 1683±4, 2231±4, 2868±4, and 2958±4 cm⁻¹; or
wherein the FT-Raman spectrum has characteristic peaks at about 155±4, 239±4, 288±4, 347±4, 402±4, 467±4, 538±4, 605±4, 672±4, 748±4, 839±4, 913±4, 1009±4, 1100±4, 1175±4, 1286±4, 1326±4, 1374±4, 1434±4, 1512±4, 1609±4, 1664±4, 1768±4, 1864±4, 1898±4, 1973±4, 2070±4, 2235±4, 2272±4, and 2390±4 cm⁻¹.

78. A process for preparing the crystalline anhydrous febuxostat (Form VIII) according to claim 74, comprising the steps of:
(a) heating febuxostat to melt under vacuum; and
(b) cooling the melted febuxostat obtained in step (a), so as to provide crystalline anhydrous febuxostat (Form VIII),
wherein the cooling in step (b) is selected from fast cooling and slow cooling.
